(19) United States
(12) Patent Application Publication
(10) Pub. No.: US 2012/0186487 A1
(43) Pub. Date: Jul. 26, 2012

(54) INORGANIC CARRIER MATERIALS CONTAINING HETEROCYCLIC 3-RING COMPOUNDS

(75) Inventor: Andreas Böttcher, Koln (DE)

(73) Assignee: LANXESS DEUTSCHLAND GMBH, Leverkusen (DE)

(21) Appl. No.: 13/377,317

(22) PCT Filed: Jun. 11, 2010
(86) PCT No.: PCT/EP10/58242

§ 371 Date: Apr. 12, 2012

(30) Foreign Application Priority Data
Jun. 12, 2009 (EP) 091620601.0

(51) Int. Cl. C09D 5/00; C07D 203/06

(52) U.S. Cl. 106/18.32; 548/955; 523/122

(57) ABSTRACT

Inorganic carrier material, comprising at least one adsorptively or covalently bound heterocyclic 3-membered-ring compound.

The invention relates to inorganic carrier material comprising heterocyclic 3-membered-ring compounds, to the preparation thereof and to the use thereof for stabilizing iodine-containing compounds, and to binder formulations comprising them and to the use thereof for the protection of industrial materials.

Iodine-containing biocides are used for providing industrial materials, coating materials being an example, with protection from infestation, decomposition, destruction and visual alteration by fungi, bacteria and algae, preferentially by fungi. Furthermore, iodine-containing biocides, both alone and in combination with biocides from other classes of active ingredient, are used as components of biocidally active materials protection compositions such as wood preservatives. Besides iodoalkynyl compounds, the active ingredients used here include compounds in which one or more atoms of iodine are attached to double-bond systems, but also to singly bonded carbon atoms.

A behaviour common to many iodine-containing biocides is that on exposure to light even in bulk or as a component of an industrial material (coating material, for example) they lead to yellowing with breakdown of the active compound. This feature hinders or prevents the use of iodine-containing biocides in materials having such sensitivity, such as in light-coloured or white coating materials, for example. For the IPBC, these destable properties are for example described in WO00/16628.

Many iodine-containing biocides, particularly iodoalkynyl compounds, are destroyed with particular rapidity by metal compounds. This fact prevents iodoalkynyl compounds, for example, from being used in solvent-based coating materials, such as paints, varnishes and stains, for example, or in biocidal preservatives, such as wood preservative primers, wood preservative impregnation systems and wood preservative stains, for example, since these alkyd-based coating and preservation systems are regularly equipped with metal compounds. In such systems, transition metal compounds, examples being cobalt, lead, manganese and vanadium octoates, function as dryers (siccatives) for the alkyd resin-containing binder system. Moreover, transition metal compounds are also used as pigments, and in some cases have destructive properties comparable with the siccatives.

In the solvent-based systems referred to above, there are, in addition to the dryers, a series of further ingredients which, to different degrees, lead to breakdown of iodine-containing biocides. Whereas the destabilizing effect is still relatively weak with the solvents that are customarily used, the other customary components of a paint formulation, such as process additives, plasticizers, colour pigments, anti-settling agents, thixotropic agents, corrosion inhibitors, anti-skinning agents and binders, for example, exhibit more or less strongly pronounced destabilizing effects.

As well as in the solvent-based systems described above, problems also attend the use of iodine-containing biocides in certain water-based industrial materials (e.g. coating materials and preservatives such as wood preservative stains and primers). Where the film formation and film hardening of a water-based coating material is based, for example, on the oxidative crosslinking of water-soluble or emulsified alkyd resins, transition metal compounds are employed as siccatives in these systems as well, and their use is accompanied by destruction of the iodine-containing biocides present.

There are already methods known for preventing the degradation of halopropargyl compounds in transition-metal-containing, solvent-based alkyd-resin paints. WO 98/22543, for example, describes the addition of chelating reagents.

Also known are transition-metal-containing, solvent-based alkyd-resin paints where halopropargyl compounds are stabilized by means of organic epoxides (cf. WO 00/16628).

Moreover, there are already descriptions of methods of suppressing the light-induced degradation of active antifungal compounds, such as iodopropargyl butylcarbamate, by addition of tetraalkylpiperidine compounds and/or UV absorbers (cf. EP-A 0083308).

According to WO 2007/028527, iodine-containing biocides are stabilized with 2-(2-hydroxyphenyl)benzotriazoles.

Addition of epoxy compounds is said to reduce the discoloration of iodoalkyne compounds, such as IPBC (cf. U.S. Pat. No. 4,276,211 and U.S. Pat. No. 4,297,258).

Furthermore, there are descriptions of synergistic mixtures of epoxides with UV absorbers (cf. WO 99/29176) and with benzylidene camphor derivatives (cf. U.S. Pat. No. 6,472,424), which likewise exhibit reduced yellowing.

WO 2007/101549, moreover, describes the stabilization of iodine-containing biocides by means of azole compounds.

The stabilizing action of the aforementioned stabilizers, however, is not always sufficient, and carries performance disadvantages. Thus, in particular, the drying times of the paints are markedly prolonged, and in many cases this is unacceptable to the user. Moreover, the inhibition of discoloration is not always sufficient.

Surprisingly, it has now been found that the use of specific, heterocyclic 3-membered-ring compounds, particularly aziridines, makes it possible to provide iodine-containing biocides, particularly in solvent-based and water-based systems, with protection against both chemical and light-induced degradation, and hence to prevent the above-described disadvantages of unstabilized iodine-containing compounds, such as alterations to colour and loss of active compound/activity. It has been found, moreover, that using carrier materials provided in this manner to stabilize iodine-containing biocides in the aforementioned systems engenders no performance disadvantages, such as the prolongation of the drying time of a coating system, for example.

Aziridine compounds are employed, for example, in US2004/0077783 A1 as a constituent of polymerization initiators which as further constituents comprise organoborane compounds, carrier materials and optionally fillers. The latter, however, are present, as a concomitant of the preparation procedure, in the form of mixtures with the other components, and not as carrier materials surface-modified with aziridine.

Through the use of adsorptively or chemically bonded aziridines on an inorganic carrier material, the specific properties of liquid, iodine-containing formulations, examples being solutions and dispersions, such as, for example, low concentration and hence unnecessary transport of solvent, are improved still further. As compared with iodine-containing solutions, this form possesses, in particular, stability advantages, particularly in storage, preferably at elevated temperatures.

The invention accordingly provides an inorganic carrier material comprising at least one adsorptively or covalently bound heterocyclic 3-membered-ring compound.

The carrier material of the invention is preferably solid at room temperature.

Inorganic carrier materials to use that are contemplated are preferably silicas such as, for example, precipitated silicas, such as silica gels, mesoporous silicates, xerogels, aerogels, fumed silicas, silicas modified with organic, inorganic or organometallic radicals, examples being dichlorodialkylsilane-modified silicas, kieselghur, porosils, examples being zeosils, clathrasils or dealuminized zeolites, aluminosilicates, zeolites, natural or synthetic tectosilicates, natural silicates such as, for example, vermiculite, mica or pyrogenic metal oxides, for example TiO₂, including pyrogenic mixed metal oxides.

Preference is given to fumed silicas, more particularly hydrophilic or hydrophobic silicas, examples being those in commerce under the name Aerosil® from Evonik-Degussa, with the product Aerosil® 200 being particularly preferred.

It is also preferred, if the heterocyclic 3-membered-ring compound is an aziridine, for the inorganic carrier material to contain no organoborane.

Likewise preferred are hydrophilic or hydrophobic precipitated silicas, more particularly hydrophilic or hydrophobic precipitated silicas, examples being those in commerce under the name Sipernat® from Evonik-Degussa. In this case, the products Sipernat® 22S and Sipernat® 50S are particularly preferred.

The carrier materials of the invention preferably have a particle size of 0.001 to 1000 μm, more particularly of 0.005 to 500 μm.

The carrier materials of the invention are preferably notable for the capacity to absorb liquids, measured in terms of their DBP absorbancy (DBP=dibutyl phthalate; see DIN 53601 or ISO 4656) in grams per 100 g of carrier material. The DBP absorbency of the carrier materials of the invention is preferably 0.1 to 800 g/100 g, more preferably 1 to 500 g/100 g of carrier material.

The carrier materials of the invention preferably possess a specific surface area [m²/g], determined in a method based on that of Brunauer, Emmett and Teller (BET surface area; J. Am. Chem. Soc. 60, 309 (1938)) in accordance with ISO 5794/1 (Annex D), of 1 to 1200 m²/g, more preferably of 50 to 900 m²/g.

In the below-described preparation processes of the carrier materials of the invention, which in particular are adsorber materials, the covering or impregnating of the carrier material with the heterocyclic 3-membered-ring compound was accomplished adsorptively or by formation of covalent bonds. The proof that a heterocyclic 3-membered-ring compound is adsorbed or bound to the inorganic carrier material in the manner of the invention is generally furnished by the analytical detection of the heterocyclic 3-membered-ring groups, the aziridine groups for example, by means of suitable methods, an example being solid-state NMR (MAS-NMR). If a heterocyclic 3-membered-ring compound can be detected in the course, for example, of the MAS-NMR determination of the inorganic carrier material of the invention, it is an inventive inorganic carrier material for the purposes of the present invention.

Heterocyclic 3-membered-ring compounds that are contemplated include preferably those with a 3-membered-ring function which have O, NR, S or Se as a heteroatom in the 3-membered ring, where R is hydrogen or an organic radical. Preferred heterocyclic 3-membered-ring compounds are epoxides or aziridines, more particularly aziridines.

Aziridines contemplated are those which comprise one or more aziridine groups.

Preference is given, for example to aziridine compounds of the formula (I)

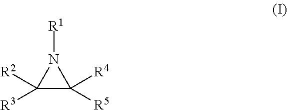

where
R¹ is hydrogen, alkyl or cycloalkyl, each of which are unsubstituted or substituted and/or mono- or polyethylenically unsaturated, or in each case substituted or unsubstituted fullerenyl, aryl, alkoxy, alkoxycarbonyl, arylcarbonyl, alkanoyl, carbamoyl or oxomethylene,
R², R³, R⁴ and R⁵ independently of one another have the same definition as R¹ and additionally independently are halogen, hydroxyl, carboxyl, alkylsulphonyl, arylsulphonyl, nitrile, isonitrile or the radicals
R² and R⁴ or R³ and R⁵, together with the carbon atoms to which they are attached, form a 5- to 10-membered carbocyclic ring which is unsubstituted or substituted and/or mono- or polyethylenically unsaturated.

Monofunctional aziridines of the formula (I) that are contemplated are, for example, those in which R² and R⁴ or R³ and R⁵, together with the carbon atoms to which they are attached, form a 5- to 10-membered carbocyclic ring which is unsubstituted or substituted and/or mono- or polyethylenically unsaturated.

These are, more particularly, those of the formula (II)

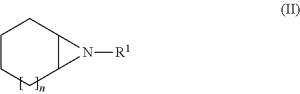

where the carbocyclic ring is unsubstituted or substituted by one or more substituents selected from the group consisting of halogen, hydroxyl, oxo, carboxyl, alkylsulphonyl, arylsulphonyl, nitrile, isonitrile, alkyl or cycloalkyl, each of which is unsubstituted or substituted and/or mono- or polyethylenically unsaturated, or substituted or unsubstituted fullerenyl, aryl, alkoxy, alkoxycarbonyl or alkanoyl, and
n is a number from 0 to 6, preferably 0 to 1.

Likewise preferred are those monofunctional aziridine compounds of the formula (I) in which R¹ is a radical of the formula

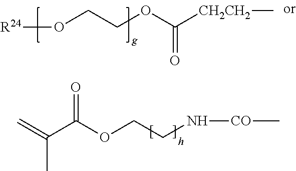

in which
R²⁴ is —H or alkyl, preferably —H, —CH₃, —C₂H₅, more preferably —CH₃, —C₂H₅,
g is a number from 1 to 4, preferably 1 to 3, more preferably 1 to 2,
h is a number from 1 to 11, preferably 1 to 5 and more preferably 1 to 3,
   and the remaining radicals have the above definition.

More particular preference is given to those compounds of the formula (I) which conform to the compound of the formula (III) or (IV),

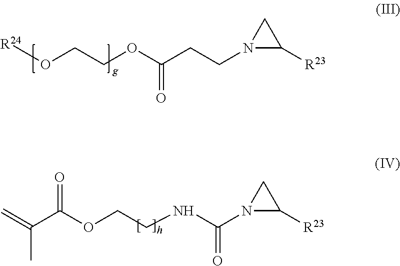

where
R²³ is —H or alkyl, preferably —H or —CH₃, more preferably —CH₃,
R²⁵ is —H or alkyl, preferably —H or —CH₃, more preferably —CH₃, and the remaining radicals have the above definition.

Particularly preferred aziridines are those having two or more aziridine functions. Examples include compounds of the formula (V)

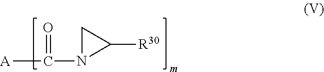

in which
A is an m-valent aliphatic, cycloaliphatic or aromatic radical, which is optionally substituted,
m is a number from 2 to 5, more particularly 2 to 3, and
R³⁰ for each m unit is in each case independently hydrogen or C₁-C₄ alkyl, more particularly CH₃ or CH₂CH₃.

Where m is 2, A is preferably C₂-C₁₀ alkylene,

more particularly

—((CH₂)₆)—, —C(CH₃)₂CH₂C(CH₃)₂CH₂— or

—C(CH₃)₂CH₂CH(CH₃)CH₂—, or

is a phenylene, more particularly the bivalent radical of the formula

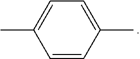

If m is 3, A is preferably the trivalent radical of the formula

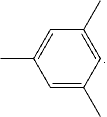

Preferred compounds of the formula (V) are those conforming to the formulae (Va)-(Vd).

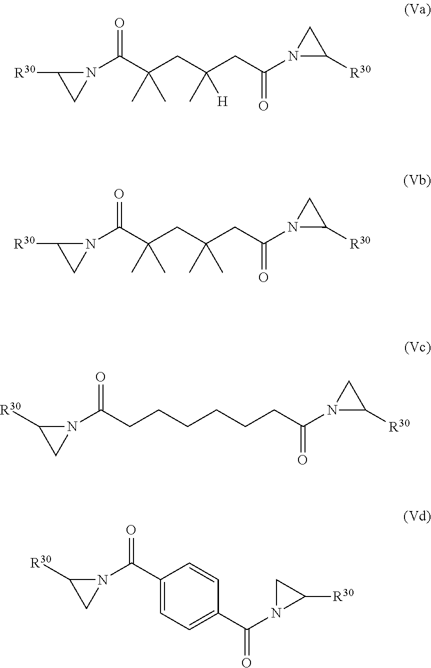

Likewise preferred as polyfunctional aziridine compounds are Michael adducts of optionally substituted ethylenimine with esters of polyhydric alcohols with α,β-unsaturated carboxylic acids and the adducts of optionally substituted ethylenimine with polyisocyanates.

Suitable alcohol components are, for example, trimethylolpropane, neopentylglycol, glycerol, pentaerythritol, 4,4′-isopropylidenediphenol, 4,4′-methylenediphenol and polyvinyl alcohols. Examples of suitable α,β-unsaturated carboxylic acids include acrylic acid and methacrylic acid, crotonic acid and cinnamic acid. Particular preference is given to acrylic acid. The corresponding polyhydric alcohols of the α,β-unsaturated carboxylic esters may optionally be alcohols which have been extended on their OH functions in some cases completely with alkylene oxides, singly or multiply. These may be, for example, the aforementioned alcohols extended singly or multiply with alkylene oxides. In this respect, reference is also made to U.S. Pat. No. 4,605,698, the disclosure content of which is included by reference in the present invention. Alkylene oxides which are particularly suitable in accordance with the invention are ethylene oxide and propylene oxide.

Examples of polyisocyanates suitable for reaction with optionally substituted ethylenimine are those specified at page 4 lines 33-35 of WO 2004/050617.

Examples of aziridines that are suitable in accordance with the invention are those specified at page 3 lines 29-34 of WO 2004/050617.

Preference is likewise given to those aziridines of the kind described, for example, in U.S. Pat. No. 3,225,013 (Fram), U.S. Pat. No. 4,490,505 (Pendergrass) and U.S. Pat. No. 5,534,391 (Wang).

Likewise preferred are those aziridines of the formula (I) which possess at least three aziridine groups, such as, for example, trimethylolpropane tris[3-(1-aziridinyl)propionate], trimethylolpropane tris[3-(2-methyl-1-aziridinyl)propionate], trimethylolpropane tris[2-aziridinylbutyrate], tris(1-aziridinyl)phosphine oxide, tris(2-methyl-1-aziridinyl)phosphine oxide, pentaerythritol tris-[3-(1-aziridinyl)propionate] and pentaerythritol tetrakis-[3-(1-aziridinyl)propionate].

Of these, preference is given particularly to trimethylolpropane tris[3-(1-aziridinyl)propionate], trimethylolpropane tris[3-(2-methyl-1-aziridinyl)propionate], trimethylolpropane tris[2-aziridinylbutyrate], pentaerythritol tris-[3-(1-aziridinyl)propionate] and pentaerythritol tetrakis-[3-(1-aziridinyl)propionate].

Particularly preferred are trimethylolpropane tris[3-(1-aziridinyl)propionate], trimethylolpropane tris[3-(2-methyl-1-aziridinyl)propionate] and pentaerythritol tetrakis-[3-(1-aziridinyl)propionate].

Likewise preferred are polyfunctional aziridines of the formula (VI)

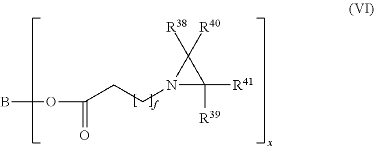

in which
B is the radical of an aliphatic polyol which contains at least x OH functions, where x OH functions are substituted by the radical of the above brackets,
f is a number from 0 to 6, more particularly from 1 to 3,
x is a number greater than or equal to 2, and more particularly is 2 to 500 000, and
R³⁸, R³⁹, R⁴⁰ and R⁴¹ have the identical meaning as the radicals R²-R⁵ in the formula (I).

Particularly preferred aziridines of the formula (VI) are those in which x is 3 or 4 and B is a trebly or quadruply OH-functional polyol.

Particularly preferred aziridines of the formula (VI) are those conforming to the formulae (VIa)-(VIc)

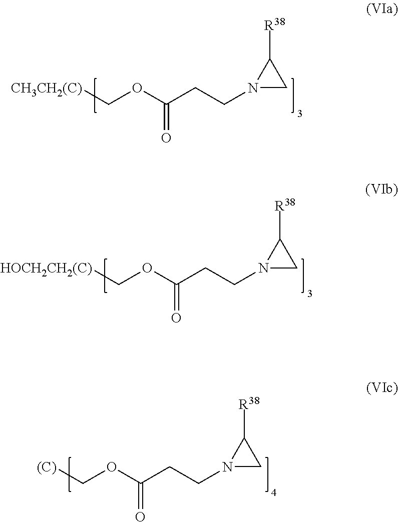

in which
R³⁸ is hydrogen or CH₃.

A particularly preferred product is the aziridine compound of the formula (VIa), with R³⁸=methyl, also known as Crosslinker CX-100 from DSM, and the hardener product “Corial Härter AN” from BASF, which comprises the aziridine of the formula (VIa) with R³⁸=hydrogen.

Epoxides contemplated include all compounds which comprise one or more oxirane rings.

Preference is given, for example, to epoxides of the general formula (VII):

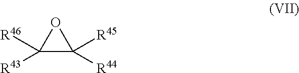

in which
R⁴³, R⁴⁴, R⁴⁵ and R⁴⁶ independently of one another are hydrogen, alkyl or cycloalkyl, each of which is unsubstituted or substituted and/or mono- or polyethylenically unsaturated, or in each case substituted or unsubstituted fullerenyl, aryl, alkoxy, aryloxy, alkanoyl, alkoxycarbonyl, arylcarbonyl, alkanoyl, carbamoyl or oxomethylene, halogen, hydroxyl, carboxyl, alkylsulphonyl, arylsulphonyl, nitrile or isonitrile
or the radicals
R⁴³ and R⁴⁴ or R⁴⁵ and R⁴⁶, together with the carbon atoms to which they are attached, form a 5- to 10-membered carbocyclic ring which is unsubstituted or substituted and/or mono- or polyethylenically unsaturated.

Preferred “alkyl” is a linear or branched alkyl radical having 1 to 20, preferably 1 to 12, carbon atoms. Examples of alkyl radicals according to the invention are methyl, ethyl, n-propyl, isopropyl, n-butyl, isobutyl, sec-butyl, tert-butyl, pentyl, iso-amyl, hexyl, octyl, etc. The aforementioned alkyl radicals may preferably be substituted by the following radicals: alkoxy, preferably C₁-C₁₂ alkoxy, nitro, monoalkylamino, preferably C₁-C₁₂ monoalkylamino, dialkylamino, preferably di[C₁-C₁₂]alkylamino, cyano, halo, haloalkyl, preferably trifluoromethyl, alkanoyl, aminocarbonyl, monoalkylaminocarbonyl, dialkylaminocarbonyl, alkylamido, preferably C₁-C₁₂ alkylamido, alkoxycarbonyl, preferably C₁-C₁₂ alkoxycarbonyl, alkylcarbonyloxy, preferably C₁-C₁₂ alkylcarbonyloxy, aryl, preferably phenyl, or aryl substituted by halogen, C₁-C₁₂ alkyl or C₁-C₁₂ alkoxy, or heterocyclic 3-membered-ring groups, more particularly aziridine groups or epoxy groups, or substituents, such as those specified above, which contain these groups.

The radicals R⁴³, R⁴⁴, R⁴⁵ and R⁴⁶ are preferably, in each case independently, hydrogen or aralkyl, aryloxyalkyl, alkoxyalkyl such as epoxydialkoxyalkyl, e.g. 2,3-epoxy-1-propoxyethoxymethyl, 2,3-epoxy-1-butoxyethoxyethyl or 3,4-epoxy-1-butoxyethoxyethyl, or are the radical of the formula

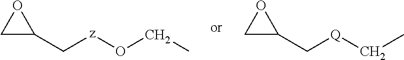

where
Z is unbranched or branched C₁-C₁₀ alkylene, more particularly propylene, butylene, pentylene, hexylene, or heptylenes, halogenated unbranched or branched C₁-C₁₀ alkylene such as, for example, 2,2-dichloromethylpropylene
and
   Q is C₁-C₄ alkylene, carbonylarylcarboxyl such as, for example, carbonylphenylcarboxyl.

The preferred epoxides include the compounds in which
R⁴⁶ is hydrogen, alkoxy, alkyl or 2,3-epoxy-1-propoxyethoxymethyl,
R⁴³ is hydrogen or alkyl and
R⁴⁴ and R⁴⁵ are hydrogen,
   and additionally R⁴³ and R⁴⁴ or R⁴⁵ and R⁴⁶, together with the carbon atoms to which they are attached, form a 5- to 10-membered carbocyclic ring which is unsubstituted or substituted and/or mono- or polyethylenically unsaturated.

The particularly preferred epoxides include glycidyl ethers. These compounds are characterized in that they comprise one or more 2,3-epoxypropanoxy groups and may be represented by the general formula (VIII):

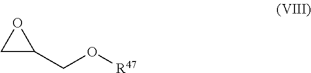

where
R⁴⁷ has the same definition as R⁴³, R⁴⁴, R⁴⁵ or R⁴⁶.

Many of these glycidyl ethers are obtainable commercially. Suitability is possessed in principle by all glycidyl ethers, more particularly those preparable by reacting 1-chloro-2,3-epoxypropane with alcohols, or by reacting glycidyl alcohol with suitable electrophiles, examples being halides.

Also particularly preferred are epoxide-containing products obtainable by reacting epichlorohydrin (1-chloro-2,3-epoxypropane) with polyhydric alcohols, more particularly polyhydric phenols such as, for example, bisphenol A, including oligomeric and polymeric reaction products. Particularly preferred are those having an average molar weight of less than 2000 g/mol, more particularly less than 1000 g/mol.

The preferred epoxides also include the following compounds:

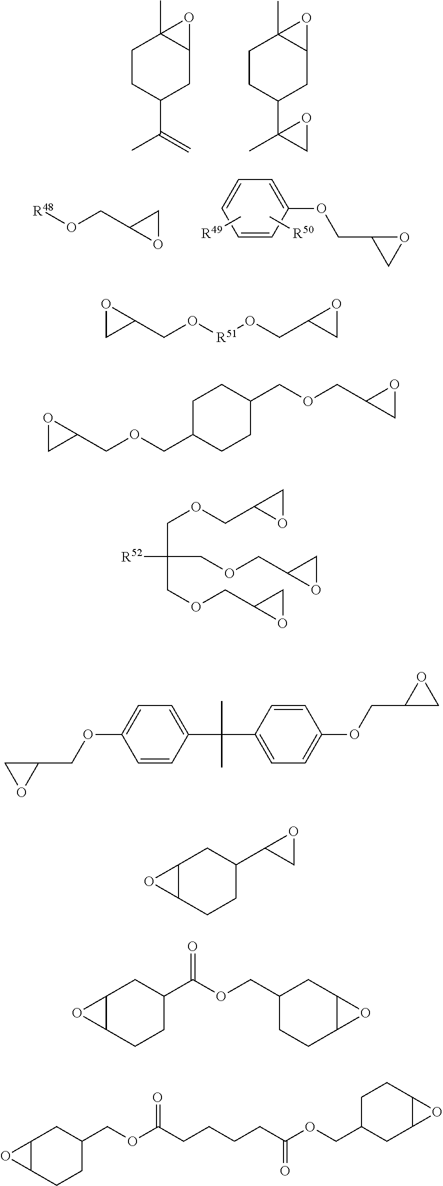

where
R⁴⁸ is C₁-C₂₀ alkyl
R⁴⁹ is H, alkyl or substituted alkyl,
R⁵⁰ is halogen,
R⁵¹ is C₁-C₂₀ alkyl and
R⁵² is H, C₁-C₂₀ alkyl, preferably methyl or ethyl.

The inorganic carrier material of the invention may further comprise additional compounds, examples being solvents such as, for example, esters of mono- or polybasic carboxylic acids (e.g. mixtures comprising diisobutyl adipate, diisobutyl glutarate, diisobutyl succinate), preferably VOC-free or low-VOC solvents, with VOCs (volatile organic compounds) being volatile organic compounds having a boiling point of less than 250° C.; emulsifiers such as, for example, castor oil ethoxylates; dispersing assistants such as, for example, polyvinyl alcohols; chelating reagents as specified in WO 98/22543, for example; one or more stabilizers from the series of the antioxidants, free-radical scavengers, UV stabilizers and/or UV absorbers (for examples, see below). In many cases, synergistic effects are observed here. The loading of the inorganic carrier material for these compounds as well may take place preferably in analogy to processes a)-d) (see above).

The carrier materials of the invention comprise preferably

0.001% to 80%, preferably 0.005% to 60%, more particularly 0.01% to 50%, by weight, of at least one heterocyclic 3-membered-ring compound, more particularly an aziridine compound, and
20%-99.999%, preferably 40%-99.995%, more preferably 50-99.99%, by weight, of at least one inorganic carrier material.

As preferred further constituents, the inorganic carrier material of the invention contains 0.0001% to 8%, preferably 0.0005% to 6%, more particularly 0.001% to 5%, by weight, of emulsifiers. The inorganic carrier material of the invention may further contain 0.1% to 15%, preferably 0.5% to 10%, more particularly 1% to 6%, by weight of a dispersing assistant.

The invention additionally relates to the use of aziridines for preparing aziridine-containing inorganic carrier materials.

The invention further relates to a process for preparing the inorganic carrier materials of the invention which is characterized in that
a) at least one—preferably one—liquid heterocyclic 3-membered-ring compound, more particularly aziridines, or solutions of at least one preferably liquid heterocyclic 3-membered-ring compound in an organic solvent, preferably in a VOC-free or low-VOC solvent, is or are mixed with an inorganic carrier, or
b) at least one heterocyclic 3-membered-ring compound, more particularly aziridines, is or are dissolved in an organic solvent and mixed with an inorganic carrier material, optionally with addition of suitable dispersing assistants and the inorganic carrier material loaded with at least one heterocyclic 3-membered-ring compound is isolated, or
c) an emulsion comprising at least one heterocyclic 3-membered-ring compound, more particularly aziridines, water, at least one organic solvent and optionally emulsifiers is mixed with an inorganic carrier material, and the inorganic carrier material loaded with at least one heterocyclic 3-membered-ring compound is isolated, or
d) an aqueous dispersion of an inorganic carrier material, comprising optionally dispersing assistants, is mixed with an emulsion comprising at least one heterocyclic 3-membered-ring compound, more particularly aziridines, water, at least one organic solvent and optionally emulsifiers, and the inorganic carrier material loaded with at least one heterocyclic 3-membered-ring compound is isolated,
   where the loaded carrier material obtained after steps b) to d) is isolated preferably by complete or partial removal of the solvent, more particularly by
   filtration and optionally subsequent drying or
   evaporation of the solvent, for example by fluidized-bed drying, spray drying or rotary evaporation optionally under reduced pressure.

Spray drying is a gentle process in which the temperatures that arise act on the slurry only for a very short time and, moreover, the applied inlet temperatures of approximately 160° C. do not affect the product directly, owing to the heat of evaporation of the water.

Preference is given to inorganic carrier materials obtainable by the process of the invention, preferably when the drying is spray drying, the materials being as described above.

In the case of process alternative a), the compounds in question are preferably heterocyclic 3-membered-ring compounds which are liquid at room temperature (22° C.), with, in particular, paste-like compositions or dry powders being formed.

In the case of process alternative b), preferred dispersants that are suitable are nonionic and anionic emulsifiers.

Particular preference is given to carrier materials, obtainable by the process of the invention, more particularly in accordance with procedure b), that comprise a dispersant, preferably anionic emulsifiers such as, for example, alkyl sulphates, alkyl ether sulphates, alkyl arylsulphonates, alkyl succinates, alkysulphosuccinates, N-alkoylsarcosinates, acyltaurates, acylisethionates, alkyl phosphates, alkyl ether phosphates, alkyl ether carboxylates, alpha-olefinsulphonates, more particularly alkali metal and alkaline earth metal salts, examples being sodium, potassium, magnesium and calcium salts, and also ammonium salts and triethanolamine salts, or ionic emulsifiers such as, for example, alkylaryl polyglycol ethers, such as polyoxyethylene octylphenol ethers, ethoxylated isooctyl-, octyl- or nonylphenol, alkylphenol or tributylphenyl polyglycol ethers, trissterylphenyl ether ethoxylates, alkylaryl polyether alcohols, isotridecyl alcohol, polyoxyethylene-fatty alcohol ethers, polyoxyethylene-fatty acid esters such as, for example, ethoxylated castor oil, polyoxyethylenealkyl ethers or polyoxypropylene, lauryl alcohol polyglycol ether acetate, sorbitol esters or block copolymers based on ethylene oxide and/or propylene oxide.

In the case of procedure c), preferred organic solvents used for the oil-in-water emulsion are preferably those specified above.

Preference is given to the preparation in accordance with process alternative d). In this case, in particular, a dispersion of the inorganic carrier material in water is prepared to start with, using preferably low shearing energy, as for example through use of a paddle stirrer.

The preparation of the O/W emulsion of the heterocyclic 3-membered-ring compound, more particularly of an aziridine, in solution in a suitable, inert organic solvent is accomplished optionally with addition of suitable emulsifiers and preferably with application of high shearing forces (e.g. Ultraturrax). The addition of the O/W emulsion of the heterocyclic 3-membered-ring compound, more particularly of the aziridine, to the dispersion of the inorganic carrier takes place preferably with a low shearing force (e.g. paddle stirrer). The loading of the inorganic carrier with the heterocyclic 3-membered-ring compound, more particularly with an aziridine, is accomplished preferably by stirring the resultant suspoemulsion for at least 12 hours under a low shearing force (e.g. paddle stirrer). The subsequent isolation of the inorganic carrier material loaded with the heterocyclic 3-membered-ring compound, more particularly with aziridines, and optionally with solvent is accomplished preferably by fluidized-bed drying or spray drying. The resultant inorganic carrier material of the invention may optionally also be deaggregated by using an annular sieve mill, and then converted into a homogeneous solids mixture with the use of a mixer, such as a Lödige mixer, for example.

Further additions may be added in the case of any of the procedures, the addition taking place in solid or liquid form or in dissolved, dispersed or emulsified form.

The process alternatives of the invention take place preferably at a temperature of 0 to 35° C., more particularly at room temperature (22° C.).

The invention further relates to the use of the carrier materials of the invention for stabilizing iodine-containing compounds, more particularly biocides.

Iodine-containing compounds contemplated are preferably iodoalkynyl compounds or compounds in which one or more iodine atoms are attached to double bonds or in which one or more iodine atoms are attached to singly bonded carbon atoms.

The iodine-containing compounds, more particularly biocides, are, for example, diiodomethyl p-tolyl sulphone, diiodomethyl p-chlorophenyl sulphone, 3-bromo-2,3-diiodo-2-propenyl alcohol, 2,3,3-triiodoallyl alcohol, 4-chloro-2-(2-chloro-2-methylpropyl)-5-[(6-iodo-3-pyridinyl)methoxy]-3(2H)-pyridazinone (CAS RN: 120955-77-3), iodofenfos, 3-iodo-2-propynyl 2,4,5-trichlorophenyl ether, 3-iodo-2-propynyl 4-chlorophenyl formal (IPCF), N-iodopropargyloxycarbonylalanine, N-iodopropargyloxycarbonylalanine ethyl ester, 3-(3-iodopropargyl)benzoxazol-2-one, 3-(3-iodopropargyl)-6-chlorobenzoxazol-2-one, 3-iodo-2-propynyl alcohol, 4-chlorophenyl 3-iodopropargyl formal, 3-iodo-2-propynyl propylcarbamate, 3-iodo-2-propynyl butylcarbamate (IPBC), 3-iodo-2-propynyl m-chlorophenylcarbamate, 3-iodo-2-propynyl phenylcarbamate, di(3-iodo-2-propynyl)hexyldicarbamate, 3-iodo-2-propynyloxyethanol ethylcarbamate, 3-iodo-2-propynyloxyethanol phenylcarbamate, 3-iodo-2-propynyl thioxothioethylcarbamate, 3-iodo-2-propynyl carbamate (IPC), 3-bromo-2,3-diiodo-2-propenyl ethylcarbamate, 3-iodo-2-propynyl n-hexylcarbamate or 3-iodo-2-propynyl cyclohexylcarbamate.

The iodine-containing compounds, more particularly biocides, are preferably 3-iodo-2-propynyl 2,4,5-trichlorophenyl ether, 3-iodo-2-propynyl 4-chlorophenyl formal (IPCF), N-iodopropargyloxycarbonylalanine, N-iodopropargyloxycarbonylalanine ethyl ester, 3-(3-iodopropargyl)benzoxazol-2-one, 3-(3-iodopropargyl)-6-chlorobenzoxazol-2-one, 3-iodo-2-propynyl alcohol, 4-chlorophenyl 3-iodopropargyl formal, 3-iodo-2-propynyl propylcarbamate, 3-iodo-2-propynyl butylcarbamate (IPBC), 3-iodo-2-propynyl m-chlorophenylcarbamate, 3-iodo-2-propynyl phenylcarbamate, di(3-iodo-2-propynyl)hexyldicarbamate, 3-iodo-2-propynyloxyethanol ethylcarbamate, 3-iodo-2-propynyloxyethanol phenylcarbamate, 3-iodo-2-propynyl thioxothioethylcarbamate, 3-iodo-2-propynyl carbamate (IPC), 3-bromo-2,3-diiodo-2-propenyl ethylcarbamate, 3-iodo-2-propynyl n-hexylcarbamate or 3-iodo-2-propynyl cyclohexylcarbamate.

With particular preference the iodine-containing compounds, more particularly biocides, are 3-iodo-2-propynyl propylcarbamate, 3-iodo-2-propynyl butylcarbamate (IPBC), 3-iodo-2-propynyl m-chlorophenylcarbamate, 3-iodo-2-propynyl phenylcarbamate, di(3-iodo-2-propynyl)hexyldicarbamate, 3-iodo-2-propynyloxyethanol ethylcarbamate, 3-iodo-2-propynyloxyethanol phenylcarbamate, 3-iodo-2-propynyl thioxothioethylcarbamate, 3-iodo-2-propynyl carbamate (IPC), 3-bromo-2,3-diiodo-2-propenyl ethylcarbamate, 3-iodo-2-propynyl n-hexylcarbamate or 3-iodo-2-propynyl cyclohexylcarbamate.

Furthermore, the particularly preferred iodine-containing compounds, more particularly biocides, are N-alkyl-iodotetrazoles, N-aryl-iodotetrazoles and N-aralkyl-iodotetrazoles, as described, for example, in (EP1773125).

The iodine-containing compounds, more particularly biocides, may be used individually or in mixtures together with two or more iodine-containing compounds, more particularly biocides. Particular preference is given to IPBC.

The carrier materials of the invention for use in accordance with the invention are suitable preferentially for stabilizing iodine-containing compounds, more particularly biocides, in binder formulations, such as in alkyd-resin-based systems such as coating materials which comprise transition metal dryers, in particular in the presence of transition metal dryers. Preferred binder formulations and transition metal dryers are described in more detail later on below.

Stabilization in the context of this specification means preferably the stabilization of iodine-containing compounds against both chemical and light-induced degradation, particularly against chemical degradation.

The carrier materials of the invention may more particularly be used for suppressing or at least retarding the chemical degradation of iodine-containing compounds, more particularly biocides in active-compound formulations, more particularly coating materials such as paints, varnishes, primers, impregnating systems, stains and other industrial materials. The carrier materials of the invention that can be used in accordance with the invention for stabilizing iodine-containing compounds, more particularly biocides, have a good stabilizing action especially in alkyd-resin-based systems such as coating materials which comprise transition metal dryers.

The stabilization is preferably realized by the iodine-containing compounds, more particularly biocides, and the carrier materials of the invention being present together in a mixture or in a medium.

It is preferred to employ a composition comprising
a) at least one inorganic carrier material of the invention and
b) at least one iodine-containing biocide.

The composition is likewise provided for the present invention.

In relation to the preferred iodine-containing compounds, more particularly biocides, and to the preferred carrier materials of the invention, the forms of preference identified above also apply in respect of the preferred compositions of the invention.

Particularly preferred compositions comprise
a) at least one inorganic carrier material comprising adsorptively or covalently bound aziridine of the formula (VI) and
b) IPBC.

The compositions of the invention contain generally 0.01%-70%, preferably 0.05%-60%, more preferably 0.1%-50%, by weight, of at least one iodine-containing biocide and comprise at least one carrier material of the invention, and so the amount of all of the heterocyclic 3-membered-ring compounds, more particularly aziridine compounds, present in the composition of the invention is 0.001%-50%, preferably 0.005%-40%, more preferably 0.01%-30%, by weight.

The composition of the invention preferably comprises the iodine-containing biocide and an aziridine compound in total at from 40% to 99% by weight.

In the context of the inventive use, the carrier material of the invention is preferably used such that the amount of all of the heterocyclic 3-membered-ring compounds, more particularly aziridines, present in the composition of the invention is generally 1% to 280% by weight, preferably 2% to 225% by weight, more particularly 5% to 110% by weight, based on the iodine-containing biocide.

It is preferred, based on the iodine-containing compound, to use 0.05 to 5, preferably 0.1 to 4, more particularly 0.25 to 2 equivalents of the heterocyclic 3-membered-ring functions, more particularly aziridine functions, that are present in the carrier material of the invention.

The composition of the invention may be present in various forms, for example as a solvent-based dispersion, water-based dispersion, solids mixture, etc.

With particular preference, the composition of the invention takes the form of a solid preparation, such as, for example, a powder or granules, more particularly having an average particle size of 50 to 2000 μm, or a compacted formulation, such as, for example, compacted powder such as, for example, pellets, tablets, etc.

Likewise with particular preference, the composition of the invention takes the form of a solvent-based dispersion, where, in order to adjust the rheological properties of the dispersion, for example, alkyd resins, modified alkyd resins, thixotropic resins, etc., and also further additives such as anti-skinning agents (antioxidants), pigments, crystallization stabilizers, etc., may be added.

Further possible ingredients of the composition of the invention that may be included are adhesives such as carboxymethylcellulose, natural and synthetic polymers in powder, particle or latex form, such as gum arabic, polyvinyl alcohol, polyvinyl acetate, and also natural phospho-lipids, such as cephalins and lecithins, and synthetic phospholipids, and also mineral and vegetable oils. Moreover, it may comprise colorants such as inorganic pigments, e.g. iron oxide, titanium oxide, Prussian Blue, and organic dyes, such as alizarin dyes, azo dyes and metal phthalocyanine dyes, and trace nutrients, such as salts of iron, manganese, boron, copper, cobalt, molybdenum and zinc, and also stabilizers known for heterocyclic 3-membered-ring compounds, in particular aziridine compounds, examples being tetramethylethylenediamine (TMEDA), triethylenediamine, and the 1,4-diazabicyclo[2.2.2]octane (DABCO) known from WO 2004/050617.

The invention further provides a process for preparing the composition of the invention.

The composition of the invention may be prepared, for example, by mixing the individual components, i.e. the carrier material and the iodine-containing compound, optionally with extenders and optionally using further adjuvants such as, for example, flow improvers, additives for increasing the electrical conductivity, additives for adjusting the dusting characteristics, etc.

For preparing the compositions of the invention in the form of solids mixtures, use is made here, optionally after pretreatment of the components that are to be mixed, using, for example, sieve mills such as the Bauermeister mill, of suitable solids mixers such as, for example, Lödige mixers, paddle mixers, tumble mixers, drum mixers with disruptors, etc. Additionally, the conversion of resultant solids mixtures into further embodiments, such as granules, compacted forms such as pillows, tablets, etc., for example is possible with use of fluid-bed granulation, use of mechanical compacting systems, optionally with addition of further additives such as binders, for example.

Another embodiment of the composition of the invention is a solvent-based dispersion comprising an iodine-containing compound, in particular IPBC, and at least one carrier material of the invention, in particular one in which the heterocyclic 3-membered-ring compound is at least one aziridine compound. In this case, the iodine-containing compound, in particular IPBC, and the carrier material of the invention are ground and dispersed (e.g. bead mill), preferably with strong shearing, in an inert organic solvent as the continuous phase (e.g. isoparaffins such as Isopar® L (isoparaffin from Exxon) or “white spirits” such as, for example, Shellsol® D60), optionally with addition of process auxiliaries and stabilizers such as, for example, rheological additives (thixotroping resins such as, for example, WorleeThix® S6358, a thixotroped alkyd resin from Worlee) and optionally anti-skinning agents such as, for example, Antiskin® 444 (from Borchers).

The activity and the spectrum of action of the compositions of the invention and/or of the iodine-containing compound employed may be increased by adding, optionally, further antimicrobial compounds, fungicides, bactericides, herbicides, insecticides or other active compounds, so as to widen the spectrum of activity or to obtain particular effects, or by using such compounds at the same time. These mixtures may possess an even broader spectrum of action.

In many cases, synergistic effects are obtained, i.e. the activity of the mixture is greater than the activity of the individual components. The following compounds, for example, are particularly favourable co-components:

triazoles such as:
azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, myclobutanil, metconazole, paclobutrazole, penconazole, propiconazole, prothioconazole, simeconazole, (±)-cis-1-(4-chlorophenyl)-2-(1H-1,2,4-triazol-1-yl)cycloheptanol, 2-(1-tert-butyl)-1-(2-chlorophenyl)-3-(1,2,4-triazol-1-yl)propan-2-ol, tebuconazole, tetraconazole, triadimefon, triadimenol, triapenthenol, triflumizole, triticonazole, uniconazole and their metal salts and acid adducts;
imidazoles such as:
clotrimazole, bifonazole, climbazole, econazole, fenapamil, imazalil, isoconazole, ketoconazole, lombazole, miconazole, pefurazoate, prochloraz, triflumizole, thiazolcar, 1-imidazolyl-1-(4′-chlorophenoxy)-3,3-dimethylbutan-2-one, and their metal salts and acid adducts;
pyridines and pyrimidines such as:
ancymidol, buthiobate, fenarimol, mepanipyrin, nuarimol, pyvoxyfur, triamirol;
succinate dehydrogenase inhibitors such as:
benodanil, carboxim, carboxim sulphoxide, cyclafluramid, fenfuram, flutanil, furcarbanil, furmecyclox, mebenil, mepronil, methfuroxam, metsulphovax, nicobifen, pyracarbolid, oxycarboxin, Shirlan, Seedvax;
naphthalene derivatives such as:
terbinafine, naftifine, butenafine, 3-chloro-7-(2-aza-2,7,7-trimethyloct-3-en-5-yne);
sulphenamides such as:
dichlofluanid, tolylfluanid, folpet, fluorofolpet, captan, captofol;
benzimidazoles such as:
carbendazim, benomyl, fuberidazole, thiabendazole or their salts;
morpholine derivatives such as:
aldimorph, dimethomorph, dodemorph, falimorph, fenpropidin, fenpropimorph, tridemorph, trimorphamid and their arylsulphonate salts such as, for example, p-toluenesulphonic acid and p-dodecylphenylsulphonic acid;
benzothiazoles such as:
2-mercaptobenzothiazole;
benzothiophene dioxides such as:
N-cyclohexyl-benzo[b]thiophenecarboxamide S,S-dioxide;
benzamides such as:
2,6-dichloro-N-(4-trifluoromethylbenzyl)benzamide, tecloftalam;
boron compounds such as:
boric acid, boric esters, borax;
formaldehyde and formaldehyde-releasing compounds such as:
benzyl alcohol mono(poly)hemiformal, 1,3-bis(hydroxymethyl)-5,5-dimethylimidazolidine-2,4-dione (DMDMH), bisoxazolidine, n-butanol hemiformal, cis-1-(3-chloroallyl)-3,5,7-triaza-1-azoniaadamantane chloride, 1-[1,3-bis(hydroxymethyl-2,5-dioxoimidazolidin-4-yl]-1,3-bis-(hydroxymethyl)urea, dazomet, dimethylolurea, 4,4-dimethyloxazolidine, ethylene glycol hemiformal, 7-ethylbicyclooxazolidine, hexahydro-S-triazine, hexamethylenetetramine, N-hydroxymethyl-N′-methylthiourea, methylenebismorpholine, sodium N-(hydroxymethyl)glycinate, N-methylolchloroacetamide, oxazolidine, paraformaldehyde, taurolin, tetrahydro-1,3-oxazine, N-(2-hydroxypropyl)aminemethanol, tetramethylolacetylenediurea (TMAD);
isothiazolinones such as:
N-methylisothiazolin-3-one, 5-chloro-N-methylisothiazolin-3-one, 4,5-dichloro-N-octylisothiazolin-3-one, 5-chloro-N-octylisothiazolinone, N-octylisothiazolin-3-one, 4,5-trimethyleneisothiazolinone, 4,5-benzoisothiazolinone;
aldehydes such as:
cinnamaldehyde, formaldehyde, glutaraldehyde, β-bromocinnamaldehyde, o-phthalaldehyde;
thiocyanates such as:
thiocyanatomethylthiobenzothiazole, methylenebisthiocyanate;
quaternary ammonium compounds and guanidines such as:
benzalkonium chloride, benzyldimethyltetradecylammonium chloride, benzyldimethyldodecylammonium chloride, dichlorobenzyldimethylalkylammonium chloride, didecyldimethylammonium chloride, dioctyldimethylammonium chloride, N-hexadecyltrimethylammonium chloride, 1-hexadecylpyridinium chloride, iminoctadine tris(albesilate);
phenols such as:
tribromophenol, tetrachlorophenol, 3-methyl-4-chlorophenol, 3,5-dimethyl-4-chlorophenol, dichlorophene, 2-benzyl-4-chlorophenol, triclosan, diclosan, hexachlorophene, methyl p-hydroxybenzoate, ethyl p-hydroxybenzoate, propyl p-hydroxybenzoate, butyl p-hydroxybenzoate, octyl p-hydroxybenzoate, o-phenylphenol, m-phenylphenol, p-phenylphenol, 4-(2-tert-butyl-4-methylphenoxy)phenol, 4-(2-isopropyl-4-methylphenoxy)phenol, 4-(2,4-dimethylphenoxy)-phenol and their alkali metal salts and alkaline earth metal salts;
microbicides with an activated halogen group such as:
bronopol, bronidox, 2-bromo-2-nitro-1,3-propanediol, 2-bromo-4′-hydroxyacetophenone, 1-bromo-3-chloro-4,4,5,5-tetramethyl-2-imidazolidinone, β-bromo-β-nitrostyrene, chloroacetamide, chloramine T, 1,3-dibromo-4,4,5,5-tetramethyl-2-imidazolidinone, dichloroamine T, 3,4-dichloro-(3H)-1,2-dithiol-3-one, 2,2-dibromo-3-nitrilepropionamide, 1,2-dibromo-2,4-dicyanobutane, halane, halazone, mucochloric acid, phenyl 2-chlorocyanovinyl sulphone, phenyl 1,2-dichloro-2-cyanovinyl sulphone, trichloroisocyanuric acid;
pyridines such as:
1-hydroxy-2-pyridinethione (and the Cu, Na, Fe, Mn, Zn salts thereof), tetrachloro-4-methylsulphonylpyridine, pyrimethanol, mepanipyrim, dipyrithion, 1-hydroxy-4-methyl-6-(2,4,4-trimethylpentyl)-2(1H)-pyridine;
methoxyacrylates or similar such as:
azoxystrobin, dimoxystrobin, fluoxastrobin, kresoxim-methyl, metominostrobin, orysastrobin, picoxystrobin, pyraclostrobin, trifloxystrobin, 2,4-dihydro-5-methoxy-2-methyl-4-[2-[[[[1-[3-(trifluoromethyl)phenyl]ethylidene]amino]oxy]methyl]phenyl]-3H-1,2,4-triazol-3-one (CAS No. 185336-79-2);
metal soaps such as:
salts of the metals tin, copper and zinc with higher fatty acids, resin acids, naphthenic acids and phosphoric acid, such as, for example, tin naphthenate, tin octoate, tin 2-ethylhexanoate, tin oleate, tin phosphate, tin benzoate, copper naphthenate, copper octoate, copper 2-ethylhexanoate, copper oleate, copper phosphate, copper benzoate, zinc naphthenate, zinc octoate, zinc 2-ethylhexanoate, zinc oleate, zinc phosphate, zinc benzoate;
metal salts such as:
salts of the metals tin, copper, zinc, and also chromates and dichromates, such as, for example, copper hydroxycarbonate, sodium dichromate, potassium dichromate, potassium chromate, copper sulphate, copper chloride, copper borate, zinc fluorosilicate, copper fluorosilicate;
oxides such as:
oxides of the metals tin, copper and zinc, such as, for example, tributyltin oxide, Cu₂O, CuO, ZnO;
oxidizing agents such as:
hydrogen peroxide, peracetic acid, potassium persulphate;
dithiocarbamates such as:
cufraneb, ferban, potassium N-hydroxymethyl-N″-methyldithiocarbamate, sodium dimethyldithiocarbamate, potassium dimethyldithiocarbamate, mancozeb, maneb, metam, metiram, thiram, zineb, ziram;
nitriles such as:
2,4,5,6-tetrachloroisophthalonitrile, disodium cyanodithioimidocarbamate;
quinolines such as:
8-hydroxyquinoline and the copper salts thereof;
other fungicides and bactericides such as:
bethoxazin, 5-hydroxy-2(5H)-furanone, 4,5-benzodithiazolinone, 4,5-trimethylenedithiazolinone, N-(2-p-chlorobenzoylethyl)hexaminium chloride, 2-oxo-2-(4-hydroxyphenyl)acetohydroxycinnamoyl chloride, tris-N-(cyclohexyldiazeniumdioxy)-aluminium, N-(cyclohexyldiazeniumdioxy)-tributyltin or its potassium salts, bis-N-(cyclohexyldiazeniumdioxy)copper, iprovalicarb, fenhexamide, spiroxamine, carpropamid, diflumetorin, quinoxyfen, famoxadone, polyoxorim, acibenzolar S-methyl, furametpyr, thifluzamide, methalaxyl-M, benthiavalicarb, metrafenon, cyflufenamid, tiadinil, tea tree oil, phenoxyethanol,
Ag, Zn or Cu-containing zeolites alone or incorporated into polymeric materials.

Very especially preferred are mixtures with

azaconazole, bromuconazole, cyproconazole, dichlobutrazol, diniconazole, diuron, hexaconazole, metaconazole, penconazole, propiconazole, tebuconazole, dichlofluanid, tolylfluanid, fluorfolpet, methfuroxam, carboxin, N-cyclohexyl-benzo[b]thiophenecarboxamide S,S-dioxide, fenpiclonil, 4-(2,2-difluoro-1,3-benzodioxol-4-yl)-1H-pyrrole-3-carbonitrile, butenafine, imazalil, N-methylisothiazolin-3-one, 5-chloro-N-methylisothiazolin-3-one, N-octylisothiazolin-3-one, dichloro-N-octylisothiazolinone, mercaptobenzothiazole, thiocyanatomethylthiobenzothiazole, thiabendazole, benzoisothiazolinone, N-(2-hydroxypropyl)aminomethanol, benzyl alcohol (hemi)formal, N-methylolchloroacetamide, N-(2-hydroxypropyl)aminemethanol, glutaraldehyde, omadine, Zn-omadine, dimethyl dicarbonate, 2-bromo-2-nitro-1,3-propanediol, bethoxazin, o-phthalaldehyde, 2,2-dibromo-3-nitrilepropionamide, 1,2-dibromo-2,4-dicyanobutane, 1,3-bis(hydroxymethyl)-5,5-dimethylimidazolidine-2,4-dione (DMDMH), tetramethylolacetylenediurea (TMAD), ethylene glycol hemiformal, p-hydroxybenzoic acid, carbendazim, chlorophen, 3-methyl-4-chlorophenol, o-phenylphenol.

Apart from with the abovementioned fungicides and bactericides, mixtures with a good efficacy are, moreover, also prepared with other active compounds:

insecticides/acaricides/nematicides:
abamectin, acephate, acetamiprid, acetoprole, acrinathrin, alanycarb, aldicarb, aldoxycarb, aldrin, allethrin, alpha-cypermethrin, amidoflumet, amitraz, avermectin, azadirachtin, azinphos A, azinphos M, azocyclotin,
Bacillus thuringiensis, barthrin, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, bioresmethrin, bioallethrin, bistrilfluoron, bromophos A, bromophos M, bufencarb, buprofezin, butathiophos, butocarboxim, butoxycarboxim,
cadusafos, carbaryl, carbofuran, carbophenothion, carbosulphan, cartap, quinomethionate, cloethocarb, chlordane, chlorethoxyfos, chlorfenapyr, chlorfenvinphos, chlorfluazuron, chlormephos, N-[(6-chloro-3-pyridinyl)methyl]-N′-cyano-N-methylethanimidamide, chlorpicrin, chlorpyrifos A, chlorpyrifos M, cis-resmethrin, clocythrin, clothiazoben, cypophenothrin, clofentezin, coumaphos, cyanophos, cycloprothrin, cyfluthrin, cyhalothrin, cyhexatin, cypermethrin, cyromazin,
decamethrin, deltamethrin, demeton M, demeton S, demeton-S-methyl, diafenthiuron, dialiphos, diazinon, 1,2-dibenzoyl-1(1,1-dimethyl)hydrazine, DNOC, dichlofenthion, dichlorvos, dicliphos, dicrotophos, difethialone, diflubenzuron, dimethoate, 3,5-dimethylphenyl methylcarbamate, dimethyl(phenyl)silylmethyl-3-phenoxybenzyl ether, dimethyl(4-ethoxyphenyl)silylmethyl-3-phenoxybenzyl ether, dimethylvinphos, dioxathion, disulphoton,
eflusilanate, emamectin, empenthrin, endosulphan, EPN, esfenvalerate, ethiofencarb, ethion, ethofenprox, etrimphos, etoxazole, etobenzanid,
fenamiphos, fenazaquin, fenbutatin oxide, fenfluthrin, fenitrothion, fenobucarb, fenothiocarb, fenoxycarb, fenpropathrin, fenpyrad, fenpyroximate, fensulphothion, fenthion, fenvalerate, fipronil, flonicamid, fluacrypyrim, fluazuron, flucycloxuron, flucythrinate, flufenerim, flufenoxuron, flupyrazofos, flufenzine, flumethrin, flufenprox, fluvalinate, fonophos, formethanate, formothion, fosmethilan, fosthiazate, fubfenprox, furathiocarb,
halofenozide, HCH, (CAS RN: 58-89-9), heptenophos, hexaflumuron, hexythiazox, hydramethylnon, hydroprene,
imidacloprid, imiprothrin, indoxycarb, iprinomectin, iprobenfos, isazophos, isoamidophos, isofenphos, isoprocarb, isoprothiolane, isoxathion, ivermectin,
kadedrin,
lambda-cyhalothrin, lufenuron,
malathion, mecarbam, mervinphos, mesulphenphos, metaldehyde, methacrifos, methamidophos, methidathion, methiocarb, methomyl, metolcarb, milbemectin, monocrotophos, moxiectin,
naled, NI 125, nicotine, nitenpyram, noviflumuron,
omethoate, oxamyl, oxydemethon M, oxydeprofos,
parathion A, parathion M, penfluoron, permethrin, 2-(4-phenoxyphenoxy)ethyl ethylcarbamate, phenthoate, phorate, phosalon, phosmet, phosphamidon, phoxim, pirimicarb, pirimiphos M, pirimiphos A, prallethrin, profenophos, promecarb, propaphos, propoxur, prothiophos, prothoate, pymetrozin, pyrachlophos, pyridaphenthion, pyresmethrin, pyrethrum, pyridaben, pyridalyl, pyrimidifen, pyriproxifen, pyrithiobac-sodium,
quinalphos,
resmethrin, rotenone,
salithion, sebufos, silafluofen, spinosad, spirodiclofen, spiromesifen, sulphotep, sulprofos,
tau-fluvalinate, taroils, tebufenozide, tebufenpyrad, tebupirimphos, teflubenzuron, tefluthrin, temephos, terbam, terbufos, tetrachlorvinphos, tetramethrin, tetramethacarb, thiacloprid, thiafenox, thiamethoxam, thiapronil, thiodicarb, thiofanox, thiazophos, thiocyclam, thiomethon, thionazin, thuringiensin, tralomethrin, transfluthrin, triarathen, triazophos, triazamate, triazuron, trichlorfon, triflumuron, trimethacarb,
vamidothion, xylylcarb, zetamethrin;
molluscicides:
fentin acetate, metaldehyde, methiocarb, niclosamide;
herbicides and algicides:
acetochlor, acifluorfen, aclonifen, acrolein, alachlor, alloxydim, ametryn, amidosulphuron, amitrole, ammonium sulphamate, anilofos, asulam, atrazine, azafenidin, aziptrotryne, azimsulphuron,
benazolin, benfluralin, benfuresate, bensulphuron, bensulphide, bentazone, benzofencap, benzthiazuron, bifenox, bispyribac, bispyribac-sodium, borax, bromacil, bromobutide, bromofenoxim, bromoxynil, butachlor, butamifos, butralin, butylate, bialaphos, benzoyl-prop, bromobutide, butroxydim,
carbetamide, carfentrazone-ethyl, cafenstrole, chlomethoxyfen, chloramben, chlorbromuron, chloroflurenol, chloridazon, chlorimuron, chlornitrofen, chloroacetic acid, chloransulam-methyl, cinidon-ethyl, chlorotoluron, chloroxuron, chlorpropham, chlorsulphuron, chlorothal, chlorthiamid, cinmethylin, cinosulphuron, clefoxydim, clethodim, clomazone, chlomeprop, clopyralid, cyanamide, cyanazine, cycloate, cycloxydim, chloroxynil, clodinafop-propargyl, cumyluron, clometoxyfen, cyhalofop, cyhalofop-butyl, clopyrasulfuron, cyclosulphamuron,
diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipopetryn, diquat, dithiopyr, diduron, DNOC, DSMA, 2,4-D, daimuron, dalapon, dazornet, 2,4-DB, desmedipham, desmetryn, dicamba, dichlobenil, dimethamid, dithiopyr, dimethametryn,
eglinazine, endothal, EPTC, esprocarb, ethalfluralin, ethidirnuron, ethofumesate, ethobenzanid, ethoxyfen, ethametsulphuron, ethoxysulphuron,
fenoxaprop, fenoxaprop-P, fenuron, flamprop, flamprop-M, flazasulphuron, fluazifop, fluazifop-P, fuenachlor, fluchloralin, flufenacet, fluometuron, fluoroglycofen, fluoronitrofen, flupropanate, fluorenol, fluridone, fluorochloridone, fluoroxypyr, fomesafen, fosamine, fosametine, flamprop-isopropyl, flamprop-isopropyl-L, flufenpyr, flumiclorac-pentyl, flumipropyn, flumioxzim, flurtamone, flumioxzim, flupyrsulphuron-methyl, fluthiacet-methyl,
glyphosate, glufosinate-ammonium
haloxyfop, hexazinone,
imazamethabenz, isoproturon, isoxaben, isoxapyrifop, imazapyr, imazaquin, imazethapyr, ioxynil, isopropalin, imazosulphuron, imazomox, isoxaflutole, imazapic,
ketospiradox,
lactofen, lenacil, linuron,
MCPA, MCPA-hydrazide, MCPA-thioethyl, MCPB, mecoprop, mecoprop-P, mefenacet, mefluidide, mesosulphuron, metam, metamifop, metamitron, metazachlor, methabenzthiazuron, methazole, methoroptryne, methyldymron, methyl isothiocyanate, methobromuron, metoxuron, metribuzin, metsulphuron, molinate, manolide, monolinuron, MSMA, metolachor, metosulam, metobenzuron,
naproanilide, napropamide, naptalam, neburon, nicosuiphuron, norflurazon, sodium chlorate,
oxadiazon, oxyfluorfen, oxysulphuron, orbencarb, oryzalin, oxadiargyl,
propyzamide, prosulphocarb, pyrazolate, pyrazosulphuron, pyrazoxyfen, pyribenzoxim, pyributicarb, pyridate, paraquat, pebulate, pendimethalin, pentachlorophenol, pentoxazone, pentanochlor, petroleum oils, phenmedipham, picloram, piperophos, pretilachlor, primisulphuron, prodiamine, profoxydim, prometryn, propachlor, propanil, propaquizafob, propazine, propham, propisochlor, pyriminobac-methyl, pelargonic acid, pyrithiobac, pyraflufen-ethyl,
quinmerac, quinocloamine, quizalofop, quizalofop-P, quinchlorac,
rimsulphuron,
sethoxydim, sifuron, simazine, simetryn, sulphosulphuron, sulphometuron, sulphentrazone, sulcotrione, sulphosate,
tar oils, TCA, TCA-sodium, tebutam, tebuthiuron, terbacil, terbumeton, terbuthylazine, terbutryn, thiazafluoron, thifensulphuron, thiobencarb, thiocarbazil, tralkoxydim, triallate, triasulphuron, tribenuron, triclopyr, tridiphane, trietazine, trifluralin, tycor, thdiazimin, thiazopyr, triflusulphuron,
vernolate.

The invention further provides a binder formulation comprising
at least one binder,
at least one iodine-containing compound, more particularly biocide, and
at least one carrier material of the invention.

The binder formulation preferably comprises the ‘iodine-containing compound, more particularly biocide’ and ‘carrier material of the invention’ components in the form of the composition of the invention. Preferred binders contemplated include oxidatively drying binders, preferably alkyd-resin-based binders, or binders which form films by means of coalescents, especially latices.

The alkyd-resin-based binders contemplated are preferably alkyd resins and modified alkyd resins.

The alkyd resins are, in general, polycondensation resins formed from polyols and polybasic carboxylic acids and/or their anhydrides, and fats, oils or free natural and/or synthetic fatty acids. The alkyd resins may optionally also be modified chemically with hydrophilic groups, especially water-soluble groups, in order that they can be used, for example, as an emulsifiable or as a water-soluble alkyd resin.

The stated polyols are preferably glycerol, pentaerythritol, trimethylolethane, trimethylolpropane and various diols such as ethane-/propanediol, diethylene glycol and neopentyl glycol.

The stated polybasic carboxylic acids and/or their anhydrides are preferably phthalic acid, phthalic anhydride, maleic anhydride, isophthalic acid, terephthalic acid, trimellitic anhydride, adipic acid, azelaic acid or sebacic acid.

The stated oils or fatty acids are generally linseed oil, oiticica oil, tung oil, soya oil, sunflower oil, safflower oil, ricinene oil, tall oil, castor oil, coconut oil, peanut oil, their fatty acids, and also synthetic saturated, unsaturated or polyunsaturated monocarboxylic acids or mixtures of these components.

The alkyd resins can optionally also be modified with, for example, natural resins, phenolic resins, acrylic resins, styrene, epoxy resins, silicone resins, isocyanates, polyamides or aluminium alkoxides.

The alkyd resins generally have a molar mass of 500 to 100 000 g/mol, preferably of 1000 to 50 000 g/mol, more particularly of 1500 to 20 000 g/mol, (determined by laser light scattering; see, for example, “Static Light Scattering of Polystyrene Reference Materials: Round Robin Test”, U. Just, B. Werthmann International Journal of Polymer Analysis and Characterization, 1999 Vol. 5, pages 195-207).

The binder formulations of the invention comprise preferably 1% to 80%, more preferably 2% to 70% and with particular preference 3% to 60% by weight of alkyd resin.

The binder formulation of the invention preferably comprises an alkyd-resin-based binder and a transition metal dryer for oxidative drying. Transition metal dryers for the purposes of this specification are more particularly transition metal compounds which accelerate the drying and curing of the alkyd-resin-based binder.

Preference is given to the salts of transition metals of groups Vb, VIb, VIIb, VIII and Ib of the chemical periodic system. These are more particularly the salts of cobalt, manganese, vanadium, nickel, copper and iron, more preferably cobalt, manganese, iron and vanadium. They need not necessarily be used alone, but instead can also be employed in combination with non-transition metal salts, such as lead, calcium or zirconium, for example.

The preferred transition metal salts are soluble in organic solvents, for example, white spirit at 20° C. in an amount of more than 10 g/l. The salts in question are preferably the salts of carboxylic acids, which have high compatibility with the alkyd resin binders and at the same time ensure sufficient solubility of the metal salt. Preference is given to using transition metal salts of fatty acids, such as oleates or linoleates, resin acids such as resinates, or salts of 2-ethylhexanoic acid (octoates). Preferred transition metal dryers are cobalt octoate and cobalt naphthenate, e.g. Octasoligen®-Cobalt 12 from Borchers.

The binder formulations of the invention preferably comprise the transition metal dryers in an amount of 0.001% to 1%, preferably 0.005% to 0.5% and very preferably 0.01% to 0.1% by weight, based in each case on binder.

In one preferred embodiment the binder formulations comprise at least one polar organic solvent, preferably a polar aprotic solvent. Examples of suitable such polar protic solvents are those such as dipropylene glycol monomethyl ether (e.g. Dowanol DPM from Dow Chemical) and also, preferably, in combination thereto, polar aprotic solvents, such as dimethylformamide and dimethyl sulphoxide, and also, for example, etherified glycols, oligoglycols and polyglycols, etherified polyols and esterified polyols, esters of monobasic and polybasic carboxylic acids, e.g. diisobutyl adipate, diisobutyl maleate, (e.g. Rhodiasolv DIB).

Particular preference is given to the binder formulation comprising

1% to 80%, preferably 2% to 70%, more preferably 3% to 60% by weight of alkyd resin binder(s),
0% to 50%, preferably 0% to 45%, more preferably 0% to 40% by weight of colour pigments,
0.01% to 5%, preferably 0.05% to 3%, more preferably 0.1% to 2% by weight of iodine-containing compound, in particular biocide,
0.001% to 5%, preferably 0.005% to 3%, more preferably 0.01% to 2% by weight of the heterocyclic 3-membered-ring compound, in particular aziridine compounds,
2% to 97% by weight of solvent(s), more particularly non-polar or polar solvents, including preferably up to 10%, more particularly 0.01% to 7.5%, by weight, based on the binder preparation, of polar aprotic solvents, and
0.001% to 3% by weight of a transition metal dryer.

Particularly preferred binder formulations of the invention are those comprising at least one alkyd resin, at least one transition metal dryer, IPBC, at least one solvent and at least one carrier material of the invention, comprising at least one aziridine compound which is bound adsorptively or covalently.

The binder formulation may further comprise fillers, anti-skinning agents, rheological additives such as, for example, anti-settling agents and thixotropic agents, further biocides such as fungicides, bactericides, anti-fouling agents and algicides, solvents, process additives, plasticizers, UV stabilizers and heat stabilizers, and also corrosion inhibitors, in customary amounts.

It is additionally possible to add further stabilizers to the binder formulations, examples being the chelating reagents specified in WO 98/22543, or other heterocyclic 3-membered-ring compounds, in particular those with a different heteroatom from that which was used in the loaded 3-membered-ring compound on the inorganic carrier material. In the case of aziridines, these are preferably the organic epoxides specified in WO 00/16628. In many cases synergistic effects are observed here.

In the context of the inventive use it is also possible, furthermore, to add one or more stabilizers from the group consisting of antioxidants, free-radical scavengers, UV stabilizers, chelators and UV absorbers, which in some cases exhibit synergistic effects.

Further UV stabilizers that may be mentioned include, by way of example, the following: sterically hindered phenols, such as

2,6-di-tert-butyl-4-methylphenol, 2-tert-butyl-4,6-dimethylphenol, 2,6-di-cyclopentyl-4-methylphenol, 2-(α-methylcyclohexyl)-4,6-dimethylphenol, 2,6-di-octadecyl-4-methylphenol or 2,6-di-tert-butyl-4-methoxymethylphenol, diethyl (3,5-di-tert-butyl-4-hydroxybenzyl)phosphonate, 2,4-dimethyl-6-(1-methylpentadecyl)phenol, 2-methyl-4,6-bis[(octylthio)methyl]phenol, 2,6-di-tert-butyl-4-methoxyphenol, 2,5-di-tert-butylhydroquinone, 2,5-di-tert-amylhydroquinone, 2,6-diphenyl-4-octadecyloxyphenol, 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 2,2′-methylenebis(6-tert-butyl-4-methylphenol), 2,2′-methylenebis-(4-methyl-6-cyclohexylphenol), 2,2′-methylenebis(4,6-di-tert-butylphenol), 2,2′-ethylidenebis(4,6-di-tert-butylphenol), 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 1,1,3-tris(5-tert-butyl-4-hydroxy-2-methylphenyl)butane, 1,3,5-tri(3,5-di-tert-butyl-4-hydroxybenzyl)-2,4,6-trimethylbenzene, isooctyl 3,5-di-tert-butyl-4-hydroxybenzylmercaptoacetate, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 1,3,5-tris[(3,5-di-tert-butyl-4-hydroxyphenyl)propionyloxyethyl]isocyanurate, dioctadecyl 3,5-di-tert-butyl-4-hydroxybenzylphosphonate, calcium monoethyl 3,5-di-tert-butyl-4-hydroxybenzylphosphonate, N,N′-di-(3,5-di-tert-butyl-4-hydroxyphenylpropionyl)hexamethylenediamine, N,N′-di-(3,5-di-tert-butyl-4-hydroxyphenylpropionyl)trimethylenediamine, N,N′-di-(3,5-di-tert-butyl-4-hydroxyphenylpropionyl)hydrazine, 3,9-bis[1,1-dimethyl-2-[(3-tert-butyl-4-hydroxy-5-methylphenyl)propionyloxy]ethyl]-2,4,8,10-tetraoxaspiro[5.5]undecane, bis[3,3-bis(4′-hydroxy-3′-tert-butylphenyl)butanoic acid]ethylene glycol ester, 2,6-bis[[3-(1,1-dimethylethyl)-2-hydroxy-5-methylphenyl]octahydro-4,7-methano-1H-indenyl]-4-methylphenol (=Wingstay L), 2,4-bis(n-octylthio)-6-(3,5-di-tert-butyl-4-hydroxyphenylamino)-s-triazine, N-(4-hydroxyphenyl)octadecaneamide, 2,4-di-tert-butylphenyl 3′,5′-di-tert-butyl-4′-hydroxybenzoate, (benzoic acid, 3,5-bis(1,1-dimethylethyl)-4-hydroxy-, hexadecyl ester), 3-hydroxyphenyl benzoate, 2,2′-methylenebis(6-tert-butyl-4-methylphenol)monoacrylate, dimethylethyl)-6-[1-[3-(1,1-dimethylethyl)-5-(1,1-dimethylpropyl)-2-hydroxyphenyl]ethyl]-4-(1,1-dimethylpropyl)phenyl ester, esters of β-(3,5-di-tert-butyl-4-hydroxyphenyl)propionic acid with monohydric or polyhydric alcohols such as, for example, with methanol, octadecanol, 1,6-hexanediol, neopentyl glycol, thiodiethylene glycol, diethylene glycol, triethylene glycol, pentaerythritol, trishydroxyethyl isocyanurate or dihydroxyethyloxalamide, esters of β-(5-tert-butyl-4-hydroxy-3-methylphenyl)propionic acid with monohydric or polyhydric alcohols such as, for example, with methanol, octadecanol, 1,6-hexanediol, neopentyl glycol, thiodiethylene glycol, diethylene glycol, triethylene glycol, pentaerythritol, trishydroxyethyl isocyanurate or dihydroxyethyloxalamide.

Hindered amines, such as

bis(1,2,2,6,6-pentamethyl-4-piperidyl)2-(3,5-di-tert-butyl-4-hydroxybenzyl)-2-butylmalonate, bis(2,2,6,6-tetramethyl-4-piperidyl)decanedioate, dimethyl succinate-1-(2-hydroxyethyl)-4-hydroxy-2,2,6,6-tetramethylpiperidine copolymer, poly[[6-[(1,1,3,3-tetramethylbutyl)amino]-1,3,5-triazine-2,4-diyl][(2,2,6,6-tetramethyl-4-piperidyl)imino]hexamethylene[(2,2,6,6-tetramethyl-4-piperidyl)imino]] (CAS No. 71878-19-8), 1,5,8,12-tetrakis[4,6-bis(n-butyl-n-1,2,2,6,6-pentamethyl-4-piperidylamino)-1,3,5-triazin-2-yl]-1,5,8,12-tetraazadodecane (CAS No. 106990-43-6), bis(1,2,2,6,6-pentamethyl-4-piperidyl)decanedioate, bis(1,2,2,6,6-pentamethyl-4-piperidyl)2-(3,5-di-tert-butyl-4-hydroxybenzyl)-2-butylmalonate, decanedioic acid, bis(2,2,6,6-tetramethyl-4-piperidinyl)ester, reaction products with tert-butyl hydroperoxide and octane (CAS No. 129757-67-1), Chimasorb 2020 (CAS No. 192268-64-7), poly[[6-morpholino-1,3,5-triazine-2,4-diyl][(2,2,6,6-tetramethyl-4-piperidinyl)imino]-1,6-hexanediyl[(2,2,6,6-tetramethyl-4-piperidinyl)imino]], poly[[6-(4-morpholinyl)-1,3,5-triazine-2,4-diyl][(1,2,2,6,6-pentamethyl-4-piperidinyl)imino]-1,6-hexanediyl[(1,2,2,6,6-pentamethyl-4-piperidinyl)imino]] (9CI), 3-dodecyl-1-(2,2,6,6-tetramethyl-4-piperidyl)pyrrolidine-2,5-dione, 3-dodecyl-1-(1,2,2,6,6-pentamethylpiperidin-4-yl)pyrrolidine-2,5-dione, 4-octadecanoyloxy-2,2,6,6-tetramethylpiperidine, poly[[6-(cyclohexylamino)-1,3,5-triazine-2,4-diyl][(2,2,6,6-tetramethyl-4-piperidinyl)imino]-1,6-hexanediyl[(2,2,6,6-tetramethyl-4-piperidinyl)imino]], 1H,4H,5H,8H-2,3a,4a,6,7a,8a-hexaazacyclopenta[def]fluorene-4,8-dione, hexahydro-2,6-bis(2,2,6,6-tetramethyl-4-piperidinyl)- (CAS No. 109423-00-9), N,N′-bis(formyl)-N,N′-bis(2,2,6,6-tetramethyl-4-piperidyl)-1,6-hexanediamine, N-(tetramethyl-4-piperidinyl)maleimide-C20-24-α-olefin copolymer (CAS No. 199237-39-3), tetrakis(1,2,2,6,6-pentamethyl-4-piperidyl) 1,2,3,4-butanetetracarboxylate, tetrakis(2,2,6,6-tetramethyl-4-piperidyl) 1,2,3,4-butanetetracarboxylate, 1,2,2,6,6-pentamethyl-4-piperidinyl tridecyl 1,2,3,4-butanetetracarboxylate, (1,2,3,4-butanetetracarboxylic acid, 2,2,6,6-tetramethyl-4-piperidinyl tridecyl ester), (2,4,8,10-tetraoxaspiro[5.5]undecane-3,9-diethanol, β,β, β′,β′-tetramethyl-, polymer with 1,2,3,4-butanetetracarboxylic acid) (CAS No. 115055-30-6), 2,2,4,4-tetramethyl-21-oxo-7-oxa-3,20-diazadispiro[5.1.11.2]heneicosane, (7-oxa-3,20-diazadispiro[5.1.11.2]heneicosane-20-propanoic acid, 2,2,4,4-tetramethyl-2′-oxo-, tetradecyl ester), (7-oxa-3,20-diazadispiro[5.1.11.2]heneicosan-21-one, 2,2,4,4-tetramethyl-20-(oxiranylmethyl)-), (propanamide, N-(2,2,6,6-tetramethyl-4-piperidinyl)-3-[(2,2,6,6-tetramethyl-4-piperidinyl)amino]-), (1,3-propanediamine, N,N″-1,2-ethanediylbis-, polymer with 2,4,6-trichloro-1,3,5-triazine, reaction products with N-butyl-2,2,6,6-tetramethyl-4-piperidinamine) (CAS No 136504-96-6), 1,1′-ethylenebis(3,3,5,5-tetramethyl-2-piperazinone), (piperazinone, 1,1′,1″-[1,3,5-triazine-2,4,6-triyltris[(cyclohexylimino)-2,1-ethanediyl]]tris[3,3,5,5-tetramethyl-), (7-oxa-3,20-diazadispiro[5.1.11.2]heneicosane-20-propanoic acid, 2,2,4,4-tetramethyl-21-oxo-, dodecyl ester), 1,1-bis(1,2,2,6,6-pentamethyl-4-piperidyloxycarbonyl)-2-(4-methoxyphenyl)ethene, (2-propenoic acid, 2-methyl-, methyl ester, polymer with 2,2,6,6-tetramethyl-4-piperidinyl 2-propenoate) (CAS No 154636-12-1), (propanamide, 2-methyl-N-(2,2,6,6-tetramethyl-4-piperidinyl)-2-[(2,2,6,6-tetramethyl-4-piperidinyl)amino]-), (D-glucitol, 1,3:2,4-bis-O-(2,2,6,6-tetramethyl-4-piperidinylidene)-) (CAS No 99473-08-2), N,N′-bis(2,2,6,6-tetramethyl-4-piperidinyl)isophthalamide, 4-hydroxy-2,2,6,6-tetramethylpiperidine, 1-allyl-4-hydroxy-2,2,6,6-tetramethylpiperidine, 1-benzyl-4-hydroxy-2,2,6,6-tetramethylpiperidine, 1-(4-tert-butyl-2-butenyl)-4-hydroxy-2,2,6,6-tetramethylpiperidine, 4-stearoyloxy-2,2,6,6-tetramethylpiperidine, 1-ethyl-4-salicyloyloxy-2,2,6,6-tetramethylpiperidine, 4-methacryloyloxy-1,2,2,6,6-pentamethylpiperidine, 1,2,2,6,6-pentamethylpiperidin-4-yl β-(3,5-ditert-butyl-4-hydroxyphenyl)propionate, 1-benzyl-2,2,6,6-tetramethyl-4-piperidinyl maleate, (di-2,2,6,6-tetramethylpiperidin-4-yl)adipate, (di-2,2,6,6-tetramethylpiperidin-4-yl)sebacate, (di-1,2,3,3,6-tetramethyl-2,6-diethylpiperidin-4-yl)sebacate, (di-1-allyl-2,2,6,6-tetramethylpiperidin-4-yl)phthalate, 1-propargyl-4-β-cyanoethyloxy-2,2,6,6-tetramethylpiperidine, 1-acetyl-2,2,6,6-tetramethylpiperidin-4-yl acetate, trimellitic acid tri(2,2,6,6-tetramethylpiperidin-4-yl)ester, 1-acryloyl-4-benzyloxy-2,2,6,6-tetramethylpiperidine, dibutyl-malonic acid di(1,2,2,6,6-pentamethylpiperidin-4-yl)ester, butyl(3,5-di-tert-butyl-4-hydroxybenzyl)malonic acid di(1,2,2,6,6-pentamethylpiperidin-4-yl)ester, dibenzylmalonic acid di(1,2,2,6,6-pentamethylpiperidin-4-yl)ester, dibenzylmalonic acid di(1,2,3,6-tetramethyl-2,6-diethylpiperidin-4-yl)ester, hexane-1′,6′-bis-(4-carbamoyloxy-1-n-butyl-2,2,6,6-tetramethylpiperidine), toluene-2′,4′-bis(4-carbamoyloxy-1-n-propyl-2,2,6,6-tetramethylpiperidine), dimethyl-bis(2,2,6,6-tetramethylpiperidine-4-oxy)silane, phenyl-tris(2,2,6,6-tetramethylpiperidine-4-oxy)silane, tris(1-propyl-2,2,6,6-tetramethylpiperidin-4-yl)phosphite, tris(1-propyl-2,2,6,6-tetramethylpiperidin-4-yl)phosphate, phenyl[bis(1,2,2,6,6-pentamethylpiperidin-4-yl)]phosphonate, di(1,2,2,6,6-pentamethylpiperidin-4-yl)sebacate, N,N′-bis(2,2,6,6-tetramethylpiperidin-4-yl)hexamethylene-1,6-diamine, N,N-bis(2,2,6,6-tetramethylpiperidin-4-yl)hexamethylene-1,6-diacetamide, 1-acetyl-4-(N-cyclohexylacetamido)-2,2,6,6-tetramethylpiperidine, 4-benzylamino-2,2,6,6-tetramethylpiperidine, N,N′-bis(2,2,6,6-tetramethylpiperidin-4-yl)-N,N′-dibutyladipamide, N,N′-bis(2,2,6,6-tetramethylpiperidin-4-yl)-N,N-dicyclohexyl(2-hydroxypropylene), N,N′-bis(2,2,6,6-tetramethylpiperidin-4-yl)-p-xylylenediamine, 4-(bis-2-hydroxyethyl)amino-1,2,2,6,6-pentamethylpiperidine, 4-(3-methyl-4-hydroxy-5-tert-butyl-benz-amido)-2,2,6,6-tetramethylpiperidine, 4-methacrylamino-1,2,2,6,6-pentamethylpiperidine, 9-aza-8,8,10,10-tetramethyl-1,5-dioxaspiro[5.5]undecane, 9-aza-8,8,10,10-tetramethyl-3-ethyl-1,5-dioxaspiro[5.5]undecane, 8-aza-2,7,7,8,9,9-hexamethyl-1,4-dioxaspiro[4.5]decane, 9-aza-3-hydroxymethyl-3-ethyl-8,8,9,10,10-pentamethyl-1-5-dioxaspiro[5.5]undecane, 9-aza-3-ethyl-3-acetoxymethyl-9-acetyl-8,8,10,10-tetramethyl-1,5-dioxaspiro[5.5]undecane, 2,2,6,6-tetramethylpiperidine-4-spiro-2′-(1′,3′-dioxane)-5′-spiro-5″-(1″,3″-dioxane)-2″-spiro-4″-(2′″,2′″,6′″,6′″-tetramethylpiperidine)-3-benzyl-1,3,8-triaza-7,7,9,9-tetramethyl-spiro[4.5]decane-2,4-dione, 3-n-octyl-1,3,8-triaza-7,7,9,9-tetramethyl-spiro-[4.5]decane-2,4-dione, 3-allyl-1,3,8-triaza-1,7,7,9,9-pentamethyl-spiro[4.5]decane-2,4-dione, 3-glycidyl-1,3,8-triaza-7,7,8,9,9-pentamethyl-spiro[4.5]decane-2,4-dione, 2-isopropyl-7,7,9,9-tetramethyl-1-oxa-3,8-diaza-4-oxyspiro[4.5]decane, 2-butyl-7,7,9,9-tetramethyl-1-oxa-3,8-diaza-4-oxyspiro[4.5]decane, 2-isopropyl-7,7,9,9-tetramethyl-1-oxa-4,8-diaza-oxyspiro[4.5]decane, 2-butyl-7,7,9,9-tetramethyl-1-oxa-4,8-diaza-3-oxyspiro[4.5]decane, bis[β-(2,2,6,6-tetramethylpiperidino)ethyl]sebacate, α-(2,2,6,6-tetramethylpiperidino)acetic acid n-octyl ester, 1,4-bis(2,2,6,6-tetramethylpiperidino)-2-butene, N-hydroxymethyl-N′-2,2,6,6-tetramethylpiperidin-4-ylurea, N-methoxymethyl-N′-2,2,6,6-tetramethylpiperidin-4-ylurea, N-methoxymethyl-N′-n-dodecyl-N′-2,2,6,6-tetramethylpiperidin-4-ylurea, O-(2,2,6,6-tetramethylpiperidin-4-yl)-N-methoxymethylurethane.

Phosphites and phosphonates, such as

tris(nonylphenyl)phosphite, tris(2,4-di-tert-butylphenyl)phosphite, bis(2,4-di-tert-butylphenyl)pentaerythritol diphosphite, bis(2,6-di-tert-butyl-4-methylphenyl)pentaerythritol diphosphite, 2,2′-methylenebis(4,6-di-tert-butylphenyl)octyl phosphite, tetrakis(2,4-di-tert-butylphenyl)[1,1′-biphenyl]-4,4′-diylbisphosphonite, 2,2′-ethylidenebis(4,6-di-tert-butylphenyl)fluorophosphite, dioctadecyl pentaerythritol diphosphonite, 2-[[2,4,8,10-tetrakis(1,1-dimethylethyl)dibenzo[d,f][1,3,2]dioxaphosphin-6-yl]oxy]-N,N-bis[2-[[2,4,8,10-tetrakis(1,1-dimethylethyl)dibenzo[d,f][1,3,2]dioxaphosphin-6-yl]oxy]ethyl]ethanamine (CAS No. 80410-33-9), bis(2,4-di-tert-butyl-6-methylphenyl)ethyl phosphite, 2,4,6-tri-tert-butylphenyl 2-butyl-2-ethyl-1,3-propanediol phosphite or bis(2,4-dicumylphenyl)pentaerythritol diphosphite,
hydroxylamines, such as
N,N-bis(2-carboxyethyl)hydroxylamine, N,N-bis(benzylthiomethyl)hydroxylamine, N,N-diethylhydroxylamine, etc.
secondary arylamines, such as
N-(2-naphthyl)-N-phenylamine, 2,2,4-trimethyl-1,2-dihydroquinoline polymer (CAS No. 26780-96-1), N-2-propyl-N′-phenyl-p-phenylenediamine, N-(1-naphthyl)-N-phenylamine, (benzenamine, N-phenyl-, reaction products with 2,4,4-trimethylpentene) (CAS No. 68411-46-1) or 4-(1-methyl-1-phenylethyl)-N-[4-(1-methyl-1-phenylethyl)phenyl]aniline.

Lactones and benzofuranones, such as

### Irganox HP 136 (CAS No. 181314-48-7)

Thioethers and thioesters, such as

distearyl 3,3-thiodipropionate, dilauryl 3,3′-thiodipropionate, ditetradecyl thiodipropionate, di-n-octadecyl disulphide.

UV absorbers, such as

(methanone, [methylenebis(hydroxymethoxyphenylene)]bis[phenyl-), (methanone, [1,6-hexanediylbis[oxy(2-hydroxy-4,1-phenylene)]]bis[phenyl-), 2-benzoyl-5-methoxyphenol, 2,4-dihydroxybenzophenone, 2,2′-dihydroxy-4-methoxybenzophenone, 2-hydroxy-4-octyloxybenzophenone, 2-hydroxy-4-dodecyloxybenzophenone, 2-(2-hydroxy-4-hexyloxyphenyl)-4,6-diphenyl-1,3,5-triazine, 2,4-bis(2,4-dimethylphenyl)-6-(2-hydroxy-4-octyloxyphenyl)-1,3,5-triazine, 2-ethoxy-2′-ethyloxalic acid bisanilide, N-(5-tert-butyl-2-ethoxyphenyl)-N′-(2-ethylphenyl)oxamide, dimethyl(p-methoxybenzylidene)malonate, 2,2′-(1,4-phenylene)bis[3,1-benzoxazin-4-one], N′-(4-ethoxycarbonylphenyl)-N-methyl-N-phenylformamidine, 4-methoxycinnamic acid 2-ethylhexyl ester, 4-methoxycinnamic acid isoamyl ester, 2-phenylbenzimidazole-5-sulphonic acid, 2-cyano-3,3-diphenylacrylic acid 2-ethylhexyl ester, 2-ethylhexyl salicylate or 3-(4-methylbenzylidene)bornan-2-one,
Chelators, such as
ethylenediaminetetraacetate (EDTA), ethylenediamine, acetylacetone, nitrotriacetic acid, ethylene glycol bis(β-aminoethyl ether)-N,N-tetraacetic acid, 2,2-bipyridine, 4,4′-dimethyl-2,2-bipyridine, 2,2′,6′,2″-terpyridine, 4,4′-diphenyl-2,2′-bipyridine, 2,2′-bipyridine-3,3′-diol, 1,10-phenanthroline, 4-methyl-1,10-phenanthroline, 5-methyl-1,10-phenanthroline, 4,7-dimethyl-1,10-phenanthroline, 5,6-dimethyl-1,10-phenanthroline, 3,4,7,8-tetramethyl-1,10-phenanthroline, 4,7-diphenyl-1,10-phenanthroline, 2,4,7,9-tetramethyl-1,10-phenanthroline, N,N,N′,N′-tetramethylethylenediamine, 2-hydroxyquinoline, 8-hydroxyquinoline, 2-hydroxy-4-methylquinaldine, 5-chloro-8-hydroxyquinoline, 5,7-dichloro-8-hydroxyquinoline, 2,4-quinolinediol, 2-quinolinethiol, 8-quinolinethiol, 8-aminoquinoline, 2,2′-biquinoline, 2-quinoxalinol, 3-methyl-2-quinoxalinol, 2,3-dihydroxyquinoxaline, 2-mercaptopyridine, 2-dimethylaminopyridine, 1,2-bis(dimethylphosphino)ethane, 1,2-bis(diphenylphosphino)ethane, 1,3-bis(diphenylphosphino)propane, 1,4-bis(diphenylphosphino)butane, polyaspartic acid or iminodisuccinate.

Iodine-containing compounds, more particularly biocides, are degraded in particular in the presence of the dryers described in more detail above. Although the strongest effects are observed in the presence of these dryers, a series of further paint components also have a destabilizing effect on iodine-containing compounds, more particularly biocides. These include organic and inorganic pigments, fillers, anti-skinning agents, rheological additives such as, for example, anti-settling agents and thixotropic agents, further compounds, particularly biocides such as fungicides, bactericides, anti-fouling agents and algicides, solvents, process additives, plasticizers, UV stabilizers and heat stabilizers, corrosion inhibitors, etc. The carrier materials of the invention also display a strongly stabilizing effect here.

The compositions of the invention, used in oxidatively drying binder preparations, and the binder preparations of the invention themselves exhibit a significant reduction in drying time as compared with unstabilized iodine-containing systems, particularly systems containing IPBC or no increase in drying time as compared with the systems not equipped with IPBC (known as blank formulations).

Therefore, without any intention on the part of the applicant to rely thereon scientifically, the mechanism which takes place is probably different from the removal of metal ions, as is described, for example, with polymers of aziridines for the removal of heavy metal ions from waste waters in DE-A1-19627909. Removal of heavy metal ions would weaken the effect of the siccatives employed for this purpose, and hence would prolong the drying time correspondingly.

The binder formulations of the invention are used preferably as coating materials, more particularly as paints, varnishes, primers, impregnating systems and stains. Accordingly, the invention also provides for the use of the binder formulations of the invention as coating materials.

The invention further provides for the use of the composition of the invention for protecting industrial materials against destruction or infestation by microorganisms.

The compositions of the invention are suitable for protecting industrial materials. Industrial materials in the present context are non-living materials which have been prepared for use in industry. The industrial materials are, for example, adhesives, sizes, paper and cardboard, textiles, leather, wood, wood-based materials, coating materials and plastics articles, cooling lubricants and other materials which may be infested or decomposed by microorganisms.

Examples of microorganisms which may bring about degradation or alteration of the industrial materials include bacteria, fungi, yeasts, algae and slime organisms. The active compounds of the invention act preferably against fungi, more particularly moulds, wood-discolouring and wood-destroying fungi (Basidiomycetes) and also against slime organisms and bacteria.

Microorganisms of the following genera may be mentioned by way of example:

Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa,
Staphylococcus, such as Staphylococcus aureus.

The invention further provides industrial materials comprising at least one iodine-containing compound, in particular biocide, and at least one inorganic carrier material of the invention.

### EXAMPLES

In the examples below, stability tests accelerated by storage at elevated temperature are carried out. The IPBC was assayed in all cases by HPLC.

### Examples 1-5

Examples 1-5 describe the preparation of inventive inorganic carrier materials with adsorptively or covalently bound aziridine compounds, and of inventive compositions comprising IPBC and such carrier materials.

### Example 1

### Inorganic Carrier Material with Aziridines I

The oil phase, consisting of 9.0 g of Crosslinker CX-100 from DSM (trimethylolpropane tris[3-(2-methyl-1-aziridinyl)propionate]) and 2.5 g of Rhodiasolv® DIB from Rhodia (mixture consisting of diisobutyl adipate, diisobutyl glutarate, diisobutyl succinate), was emulsified with an emulsifier solution of 0.575 g of Tanemul® KS from Tanatex (castor oil ethoxylate with 30 eq. EO) in 25 g of water under the action of an Ultraturrax (24 000 r*min⁻¹) for 10 minutes. The colourless emulsion obtained was metered into a dispersion of 27.0 g of Aerosil® 200 from Evonik (fumed silica with a BET surface area of 200 m²/g and a DBP absorbency of 300 g/100 g) in 300 g of water with paddle stirring, and then stirred for 24 hours.

The Aerosil loaded with the aziridine and Rhodiasolv DIB was isolated from the resultant dispersion by spray drying (Büchi B-290 spray drier, pump output 45%, N₂ flow rate 35 l*min⁻¹, inlet 160° C., outlet 73° C.). This gave 33 g of a very fine, colourless solid (84% of theory).

### Example 2

### Inorganic Carrier Material with Aziridines II

The oil phase, consisting of 18.0 g of Crosslinker CX-100 from DSM (trimethylolpropane tris[3-(2-methyl-1-aziridinyl)propionate]) and 5.0 g of Rhodiasolv® DIB from Rhodia (mixture consisting of diisobutyl adipate, diisobutyl glutarate, diisobutyl succinate), was emulsified with an emulsifier solution of 1.15 g of Tanemul® KS from Tanatex (castor oil ethoxylate with 30 eq. EO) in 50 g of water under the action of an Ultraturrax (24 000 r*min⁻¹) for 10 minutes. The colourless emulsion obtained was metered into a dispersion of 54.0 g of Aerosil® 200 from Evonik (fumed silica) in 600 g of a solution of 24 g of the dispersing assistant Mowiol® 3-85 (polyvinyl alcohol from Kuraray) in 576 g of water with paddle stirring, 600 g of water were added, and the composition was subsequently stirred for 24 hours.

The Aerosil loaded with the aziridine and Rhodiasolv® DIE was isolated from the resultant dispersion by filtration. Drying gave 75.4 g of a very fine, colourless solid (98% of theory).

### Example 3

### Inventive Solid IPBC Composition I

In a drum mixer, with addition of ceramic beads (Ø 40 mm), 90.0 g of IPBC are homogenized with 128.4 g of the carrier material from Example 2 (containing 23.2% aziridine, thus making the weight ratio of IPBC to aziridine 4:1). A fine, colourless and homogeneous powder is obtained (IPBC content 41%).

### Example 4

### Inventive Solid IPBC Composition II

In a drum mixer, with addition of ceramic beads (Ø 40 mm), 90.0 g of IPBC are homogenized with 96.9 g of the aziridine formulation from Example 2 (containing 23.2% aziridine, thus making the weight ratio of IPBC to aziridine 3:1). A fine, colourless and homogeneous powder is obtained (IPBC content 48%).

### Example 5

### Inventive Solid IPBC Composition III

In a drum mixer, with addition of ceramic beads (Ø 40 mm), 90.0 g of IPBC are homogenized with 128.4 g of the aziridine formulation from Example 1 (containing 23.03% aziridine, thus making the weight ratio of IPBC to aziridine 4:1). A fine, colourless and homogeneous powder is obtained (IPBC content 41%).

### Example 6

### Inventive IPBC Addition IV as Solvent-Based Dispersion

Metered into 75.0 g of the rheological additive Worleethix® S6358 (a thixotropied alkyd resin from Worlee) with dissolver stirring (3700 rmin⁻¹) are 8.0 g of the anti-skinning agent Antiskin® 444 (from Borchers), 80 g of IPBC, 113.1 g of the inventive carrier material from Example 2 (containing 23.2% aziridine) and 225.9 g of Isopar® L (isoparaffin from Exxon). After the end of the addition, stirring is continued in a dissolver for 40 minutes more, and the resulting preliminary dispersion is ground finely, using a bead mill.

The IPBC compositions from Example 3 and 4, respectively, are incorporated in a typical, alkyd-based coating system (alkyd stain A) in the presence of a transition metal dryer (Co) and a metal oxide pigment (iron oxide). For the equipping of the coating system, the above-mentioned compositions from Example 3 and 4, respectively, and an IPBC concentrate containing IPBC and an aziridine in a ratio of 2:1 (see Table 1) without inorganic carrier material are used in each case.

TABLE 1
IPBC/aziridine concentrate (reference II)

| | |
|---|---|
| IPBC | 30% by weight |
| Crosslinker CX-100** | 15% by weight |
| Rhodiasolv DIB* | 55% by weight |

*Mixture of diisobutyl adipate, diisobutyl glutarate, diisobutyl succinate, Rhodia.
**Trimethylolpropane-tris[3-(2-methyl-1-aziridinyl)propionate]

The formula of the alkyd stain A used is shown in Table 2.

To determine the stabilization, an accelerated ageing test is carried out. For this purpose, the equipped paint system is introduced into tightly sealing 200 ml glass bottles, with only a minimum, residual amount of air remaining in the container, and stored at 40° C. The results can be seen from Table 3.

TABLE 2
Formula of a pigmented alkyd-based stain

| | | | | Ingredients of | Ingredients of |
|---|---|---|---|---|---|
| | | Ingredients of | Ingredients of | alkyd stain A-III | alkyd stain A-IV |
| | | alkyd stain A-I | alkyd stain A-II | [%] | [%] |
| | Ingredients | [%] | [%] | (Reference I) | (Reference II) |
| Alkyd | Vialkyd VAF 4349, 80 SD 60, | 22.5 | 22.5 | 22.5 | 22.5 |
| stain A | from Cytec | | | | |
| | Polar solvent Texanol, from | 5.0 | 5.0 | 5.0 | 5.0 |
| | Eastman | | | | |
| | Rheology additive BYK E411, | 0.4 | 0.4 | 0.4 | 0.4 |
| | from BYK | | | | |
| | Shellsol D60, from Shell | 65.24 | 66.35 | 67.1 | 65.47 |
| | Chemicals | | | | |
| | MK-Solcolor red iron | 4.0 | 4.0 | 4.0 | 4.0 |
| | oxide 130M (pigment | | | | |
| | preparation), from MK | | | | |
| | Chemicals | | | | |
| | Octa-Soligen ® 69 | 0.3 | 0.3 | 0.3 | 0.3 |
| | (contains 6% Co), from | | | | |
| | Borchers | | | | |
| | IPBC composition from | 2.56¹⁾ | — | — | — |
| | Example 3 | | | | |
| | IPBC composition from | — | 1.46¹⁾ | — | — |
| | Example 4 | | | | |
| | IPBC | — | — | 0.7 | — |
| | IPBC/aziridine concentrate | — | — | — | 2.33¹⁾ |
| | (reference II) | | | | |

¹⁾corresponds in each case to 0.7% by weight IPBC, based on the stain.

From Table 3 it is clear that the inorganic carrier materials coated with a 3-membered-ring compound, in terms of the stabilization of IPBC (see stains A-I and A-II), have a significantly higher stability than the unstabilized sample A-III. There is also a significant improvement relative to the IPBC sample stabilized only with aziridine, without inorganic carrier material (stain A-IV), although stain A-IV has a greater aziridine/IPBC ratio (1/2 vs. 1/3 and 1/4, respectively).

TABLE 3
Stability of IPBC in alkyd stains A (-I) to (-IV) at 40° C.

Residual IPBC content [%],
based on the initial value

| Alkyd stain | Initial | 2 weeks | 4 weeks | 8 weeks |
|---|---|---|---|---|
| A-I | 100 | 100 | 99 | 99 |
| A-II | 100 | 100 | 99 | 98 |
| A-III¹⁾ | 100 | 96 | 52 | 0 |
| A-IV²⁾ | 100 | 100 | 80 | 0 |

¹⁾non-stabilized sample
²⁾aziridine stab. IPBC, without inorganic carrier material

### Example 8

### Use of Inventive Carrier Materials in Binder Formulations

The IPBC compositions from Examples 3, 4 and 5 are incorporated in a commercial high-build wood stain “alkyd stain B” (containing alkyd resin, white spirit, iron oxide pigment, dryer, butanone oxime, UV absorber and additives). To equip the coating system with 0.7% IPBC in each case, based on the stain, the compositions of Examples 3, 4, 5 and also unstabilized IPBC are used in each case (see Table 4):

TABLE 4

| Alkyd stain B - I | | Alkyd stain B - II | |
|---|---|---|---|
| IPBC | 1.71% by weight¹⁾ | IPBC | 1.46% by weight¹⁾ |
| Composition | | Composition | |
| from | | from | |
| Example 3 | | Example 4 | |
| Alkyd stain | 98.29% by weight | Alkyd stain B | 98.54% by weight |
| B | | | |
| Alkyd stain B - III | | Alkyd stain B - IV | |
| IPBC | 1.71% by weight¹⁾ | IPBC | 0.7% by weight |
| Composition | | unstabilized | |
| from | | | |
| Example 5 | | | |
| Alkyd stain | 98.29% by weight | Alkyd stain B | 99.3% by weight |
| B | | | |

¹⁾corresponding in each case to 0.7% by weight IPBC, based on stain

The high-build stains under investigation, each equipped with 0.7% IPBC (alkyd stain B-I to alkyd stain B-IV), were prepared by mixing the weight fractions of the alkyd stain B as indicated in Table 4 and also the stated IPBC-containing compositions.

For determining the stabilization, an accelerated ageing test is carried out. For this purpose, the equipped paint system is introduced into tightly sealing 200 ml glass bottles, with only a minimum, residual amount of air remaining in the container, and stored at 40° C. The results can be seen from Table 5, whereby only the alkyd stains B-I to B-III, equipped in accordance with the invention, exhibit no significant degradation of the IPBC after 4 weeks of storage at 40° C. For the alkyd stains I and II a good stability of IPBC is found even after 8 weeks of storage at 40° C.

TABLE 5
Stability of IPBC in alkyd stains B (-I) to (-V) at 40° C.

Residual IPBC content [%],
based on the initial value

| Alkyd stain B | Initial | 2 weeks | 4 weeks | 8 weeks |
|---|---|---|---|---|
| I | 100 | 97 | 93 | 88 |
| II | 100 | 94 | 92 | 80 |
| III | 100 | 100 | 96 | |
| IV¹⁾ | 100 | 33 | 0 | 0 |

¹⁾non-stabilized sample

What is claimed is:

1. Inorganic carrier material comprising at least one adsorptively or covalently bound heterocyclic 3-membered-ring compound.

2. Inorganic carrier material according to claim 1, characterized in that the heterocyclic 3-membered-ring compound is an epoxide or an aziridine, more particularly an aziridine.

3. Inorganic carrier material according to claim 1, characterized in that the inorganic carrier material is silicas, more particularly fumed silicas, kieselguhr, porosils, clathrasils, dealuminized zeolites, aluminosilicates, zeolites, natural or synthetic tectosilicates, natural silicates, mica or pyrogenic metal oxides.

4. Inorganic carrier material according to at claim 1, characterized in that the heterocyclic 3-membered-ring compound is an aziridine of the formula I 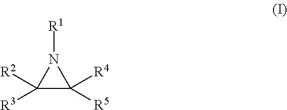
where
R¹ is hydrogen, alkyl or cycloalkyl, each of which are unsubstituted or substituted and/or mono- or polyethylenically unsaturated, or in each case substituted or unsubstituted fullerenyl, aryl, alkoxy, alkoxycarbonyl, arylcarbonyl, alkanoyl, carbamoyl or oxomethylene,
R², R³, R⁴ and R⁵ independently of one another have the same definition as R¹ and additionally independently are halogen, hydroxyl, carboxyl, alkylsulphonyl, arylsulphfonyl, nitrile or isonitrile, or the radicals
R² and R⁴ or R³ and R⁵, together with the carbon atoms to which they are attached, form a 5- to 10-membered carbocyclic ring which is unsubstituted or substituted and/or mono- or polyethylenically unsaturated.

5. Process for preparing inorganic carrier materials according to claim 1, characterized in that
a) at least one—preferably one—liquid heterocyclic 3-membered-ring compound, more particularly aziridines, or solutions of at least one heterocyclic 3-membered-ring compound in an organic solvent, is or are mixed with an inorganic carrier, or
b) at least one heterocyclic 3-membered-ring compound, more particularly aziridines, is or are dissolved in an organic solvent and mixed with an inorganic carrier material, optionally with addition of suitable dispersing assistants and the inorganic carrier material loaded with at least one heterocyclic 3-membered-ring compound is isolated, or
c) an emulsion comprising at least one heterocyclic 3-membered-ring compound, more particularly aziridines, water, at least one organic solvent and optionally emulsifiers is mixed with an inorganic carrier material, and the inorganic carrier material loaded with at least one heterocyclic 3-membered-ring compound is isolated, or
d) an aqueous dispersion of an inorganic carrier material, comprising optionally dispersing assistants, is mixed with an emulsion comprising at least one heterocyclic 3-membered-ring compound, more particularly aziridines, water, at least one organic solvent and optionally emulsifiers, and the inorganic carrier material loaded with at least one heterocyclic 3-membered-ring compound is isolated.

6. Use of inorganic carrier materials according to claim 1 for stabilizing iodine-containing compounds.

7. Composition comprising
a) at least one inorganic carrier material according to claim 1 and
b) at least one iodine-containing compound.

8. Composition according to claim 7, comprising as iodine-containing compound at least diiodomethyl p-tolylsulphone, diiodomethyl p-chlorophenyl sulphone, 3-bromo-2,3-diiodo-2-propenyl alcohol, 2,3,3-triiodoallyl alcohol, 4-chloro-2-(2-chloro-2-methylpropyl)-5-[(6-iodo-3-pyridinyl)methoxy]-3(2H)-pyridazinone (CAS RN: 120955-77-3), iodofenfos, 3-iodo-2-propynyl 2,4,5-trichlorophenyl ether, 3-iodo-2-propynyl-4-chlorophenyl formal (IPCF), N-iodopropargyloxycarbonylalanine, ethyl N-iodopropargyloxycarbonylalanine, 3-(3-iodopropargyl)benzoxazol-2-one, 3-(3-iodopropargyl)-6-chlorobenzoxazol-2-one, 3-iodo-2-propynyl alcohol, 4-chlorophenyl-3-iodopropargyl formal, 3-iodo-2-propynyl propylcarbamate, 3-iodo-2-propynyl butylcarbamate (IPBC), 3-iodo-2-propynyl m-chlorophenylcarbamate, 3-iodo-2-propynyl phenylcarbamate, di(3-iodo-2-propynyl)hexyldicarbamate, 3-iodo-2-propynyloxyethanol ethylcarbamate, 3-iodo-2-propynyloxyethanol phenylcarbamate, 3-iodo-2-propynyl thioxothioethylcarbamate, 3-iodo-2-propynyl carbamate (IPC), 3-bromo-2,3-diiodo-2-propenyl ethylcarbamate, 3-iodo-2-propynyl n-hexylcarbamate or 3-iodo-2-propynyl cyclohexylcarbamate.

9. Composition according to claim 7, comprising as iodine-containing compound at least IPBC.

10. Binder formulation comprising
at least one binder,
at least one iodine-containing compound and
at least one inorganic carrier material according to claim 1.

11. Binder formulation according to claim 10, comprising at least one oxidatively drying binder.

12. Binder formulation according to claim 10, comprising at least one transition metal dryer.

13. Binder formulation according to claim 10, comprising
1% to 80% by weight of alkyd resin binder(s),
0% to 50% by weight of colour pigments,
0.01 to 5% by weight of an iodine-containing compound,
0.001% to 5% by weight of an inorganic carrier material according to claims 1,
2% to 97% by weight of solvent(s) and
0.001% to 3% by weight of a transition metal dryer.

14. Use of the composition according to claim 7 for protecting industrial materials against destruction or infestation by microorganisms.

15. Industrial materials comprising at least one iodine-containing compound and at least one inorganic carrier material according to claim 1.

16. Use of aziridines for preparing inorganic carrier materials according to any of claims 1 to 4.
